(19) United States
(12) Patent Application Publication
(10) Pub. No.: US 2024/0148455 A1
(43) Pub. Date: May 9, 2024

(54) ROBOTIC SPINE SYSTEMS AND ROBOTIC-ASSISTED METHODS FOR TISSUE MODULATION

(71) Applicant: Relievant Medsystems, Inc., Edina, MN (US)

(72) Inventors: Brian W. Donovan, San Jose, CA (US); Samit Patel, Palo Alto, CA (US); Ray M. Baker, San Clemente, CA (US)

(21) Appl. No.: 18/550,040

(22) PCT Filed: Mar. 11, 2022
(86) PCT No.: PCT/US2022/019954

§ 371 Date: Sep. 11, 2023

Related U.S. Application Data
(60) Provisional application No. 63/161,058, filed on Mar. 15, 2021.

(51) Int. Cl. A61B 34/30; A61B 17/16; A61B 17/34; A61B 18/00; A61B 18/14; A61B 34/00; A61B 34/10; A61B 90/00

(52) U.S. Cl. CPC A61B 34/30; A61B 17/3403; A61B 17/3472; A61B 18/1487; A61B 34/10; A61B 34/74; A61B 90/37; A61B 17/1671; A61B 2018/0044; A61B 2018/00577; A61B 2034/104; A61B 2034/105; A61B 2034/107; A61B 2034/742; A61B 2090/365; A61B 2090/372

(57) ABSTRACT

Described herein are various implementations of systems and methods for accessing and modulating tissue (for example, systems and methods for accessing and ablating nerves or other tissue within or surrounding a vertebral body to treat chronic lower back pain). Assessment of vertebral endplate degeneration or defects (e.g., pre-Modic changes) to facilitate identification of treatment sites and protocols are also provided in several embodiments. Several embodiments include robotic elements for, as an example, facilitating robotically controlled access, navigation, imaging, and/or treatment.

### RELATED APPLICATION

This application claims priority to U.S. Provisional Application No. 63/161,058 filed Mar. 15, 2021, the entire content of which is hereby incorporated by reference herein.

### FIELD

Described herein are various implementations of systems and methods for modulating tissue (for example, systems and methods for ablating nerves or other tissue within or surrounding a vertebral body to treat chronic lower back pain) in which some embodiments include robotic elements for, as an example, facilitating robotically controlled access, navigation, imaging, and/or treatment. Augmented reality and virtual reality environments are also contemplated.

### BACKGROUND

Back pain is a very common health problem worldwide and is a major cause for work-related disability benefits and compensation. At any given time, low back pain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs. The back pain may be acute or chronic. Existing treatments for chronic back pain vary widely and include physical therapy and exercise, chiropractic treatments, injections, rest, pharmacological therapy such as opioids, pain relievers or anti-inflammatory medications, and surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

Robotic systems for performing surgical procedures have been developed to facilitate performance of various surgical procedures, such as inserting pedicle screws in the spine in conjunction with surgical spinal fusion procedures.

### SUMMARY

Applicant's existing technology (the Intracept® procedure by Relievant®) offers a safe and effective minimally invasive procedure that targets the basivertebral nerve for the relief of chronic vertebrogenic low back pain. As disclosed herein, several examples of systems, devices and methods provide bone access tools, additional modalities of relief for patients and/or adjunct technologies and, in certain implementations, robotic elements for, as an example, facilitating robotically controlled access, navigation, imaging, and/or treatment within vertebral bodies of the spine.

In some implementations, automated systems for accessing and/or treating tissue (such as nerves) are provided. In accordance with several examples, robotically-enabled or robotically-controlled surgical, access, and/or treatment tools may provide a high level of control and precision of movement and increased dexterity and range of motion, thereby providing increased assurance that injury will not occur to tissue not desired to be impacted. Robotic systems may be integrated or paired with augmented reality systems or devices. Robotically-controlled tools and techniques (e.g., computer-aided tools and techniques that may incorporate artificial intelligence learning and feedback) may also be used to facilitate navigation to, and surgical operation (e.g., minimally-invasive neuromodulation procedure) at, desired target treatment regions that may be difficult to access manually, thereby providing enhanced flexibility and possibilities thought not to be possible via manual human surgery. Robotically-controlled tools and techniques (e.g., computer-aided tools and techniques that may incorporate artificial intelligence learning and feedback) may further be used to facilitate capture of images pre-operatively or intra-operatively without exposing the target treatment regions to radiation or without requiring large incisions to be made. Nerve detection devices (e.g., nerve monitoring devices or nerve finders) may also be used to detect nerves along access routes that are desired to be avoided during access. Robotic or automated tools and techniques may reduce numbers of and sizes of incisions (and therefore scars), may reduce blood loss, may reduce pain, and may decrease recovery time. Robotic systems may also result in a reduction of time in waiting to rotate a C-arm imaging device back and forth between different views as may be required during manual insertion of tools in some implementations.

Treatment procedures may include modulation of nerves within or surrounding bones. The terms “modulation” or “neuromodulation”, as used herein, shall be given their ordinary meaning and shall also include ablation, permanent denervation, temporary denervation, disruption, blocking, inhibition, electroporation, therapeutic stimulation, diagnostic stimulation, inhibition, necrosis, desensitization, or other effect on tissue. Neuromodulation shall refer to modulation of a nerve (structurally and/or functionally) and/or neurotransmission. Modulation is not necessarily limited to nerves and may include effects on other tissue, such as tumors or other soft tissue.

In accordance with several examples, a method of ablating a basivertebral nerve and/or other intraosseous nerve within a vertebral body includes inserting an access assembly within a vertebral body using a robotically-controlled system integrated with an augmented reality system. The access assembly includes at least one cannula. The method further includes inserting a radiofrequency energy delivery device through the cannula to a target treatment site within the vertebral body using the robotically-controlled system and the augmented reality system, and applying power to the target treatment site using the radiofrequency energy delivery device sufficient to ablate the basivertebral nerve.

In accordance with several examples, a robotic-assisted method of ablating a basivertebral nerve and/or other intraosseous nerve(s) within a vertebral body includes providing a desired trajectory to access a target treatment site within the vertebral body to a computer-based control system of a robotic system comprising one or more robotic arms. The method further includes coupling a bone access tool to the one or more robotic arms of the robotic system and controlling insertion of the bone access tool through skin adjacent the vertebral body and into the vertebral body using the robotic system. The bone access tool may include an introducer cannula. Controlling insertion of the bone access tool includes use of an augmented reality device that includes a see-through optical head mounted display and use of one or more input devices communicatively coupled to the robotic system. The method also includes inserting a radiofrequency energy delivery device through the introducer cannula to a target treatment site within the vertebral body and applying radiofrequency energy to the target treatment site using the radiofrequency energy delivery device sufficient to ablate the basivertebral nerve and/or other intraosseous nerve(s) within the vertebral body.

The desired trajectory may be automatically generated by a computer program or may be determined by a surgeon or other clinical professional. The bone access tool may include a marker to facilitate registration of the bone access tool with a tracking system of the robotic system.

The one or more input devices may include one or more joysticks or handheld controllers, batons, or remotes. The one or more joysticks or other input devices may be communicatively coupled to the augmented reality device (directly or indirectly via the robotic system).

The desired trajectory may be displayed as a virtual image on the display of the augmented reality device. The virtual image may be overlaid or superimposed on anatomical images (e.g., 2D, 3D images) of the patient. In some implementations, the desired trajectory is configured to end at a region that includes a basivertebral nerve trunk.

The desired trajectory may be determined based on pre-operative images of spinal anatomy surrounding and including the vertebral body. The desired trajectory may be determined based on particular characteristics of the vertebral body (e.g., bone structure characteristics, such as bone density, or an anatomical identification of the vertebral body by vertebral level and number, such as L4, L5, S1, S2). In some implementations, controlling the insertion of the bone access tool includes responding to haptic feedback provided by the robotic system.

The augmented reality system may include a headset or eyewear (e.g., goggles or glasses) device configured to be worn by an operator (e.g., orthopedic surgeon or other clinician). The augmented reality device may alternatively comprise a tablet or portal that can be coupled to an arm of the robotic system or coupled to a patient operating table or stand so that the tablet or portal can be placed just above the target treatment site. The augmented reality device may include a see-through display configured to display virtual images overlaid or superimposed on top of a real-time field of view of the surgical tools and patient such that the orthopedic surgeon does not have to turn his or her head to view one or more displays or written procedural instructions outside of the procedural field of view. The virtual images may provide surgical or procedural guidance. The virtual images may include real-time video feed (e.g., fluoroscopic or computed tomography image guidance) of the target treatment site (such as a vertebral body); a planned virtual trajectory or path indicator, icon, or image; 2D, 3D or 4D pre-operative anatomical images (e.g., magnetic resonance or computed tomography images); and/or alphanumeric content (e.g., procedural instructions, cautions, warnings, alerts, real-time or previously-obtained characteristics (such as heart rate, bone density measurements, vertebral body level indicators or labels (e.g., L4, L5, S1), and/or real-time treatment parameters (e.g., impedance measurements, treatment duration timer, temperature measurements, power output measurements)).

The operator may also input text or annotations on the display of the augmented reality device using voice-activated commands or via a virtual touch-screen keyboard on the display or via one or more joysticks or other handheld controllers wirelessly coupled to the augmented reality device. The inputted text or annotations may be reproduced on other displays within a hospital operating room, ambulatory surgical center, or outpatient procedure room for others to view. The inputted text or annotations may be recorded and stored with the intra-operative video or still images (e.g., for documentation or follow-up purposes). The annotations and video or still images may be captured and stored on a cloud server system or other proprietary physical server system. The augmented reality device may also allow operators to perform procedures from remote locations over a communications network. The augmented reality device may be communicatively coupled to the robotically-controlled system so that the operator can control operation of the robotically-controlled system from an in-person location or from a remote location.

In some implementations, the robotically-controlled system includes one or more robotic arms and an operator control console including at least one processor. The system may include one or more imaging devices configured to provide feedback (e.g., based on artificial intelligence processing algorithms) to the robotically-controlled system to control insertion of the access assembly and/or the radiofrequency energy delivery device and/or to control neuromodulation parameters (e.g., ablation parameters) or determine a desired target treatment site location to facilitate optimum treatment.

The robotically-controlled system may comprise one or more robotic arms. The one or more robotic arms may have at least six degrees of freedom. The robotically-controlled system may include an operator control console comprising at least one processor. The robotically-controlled system may further include one or more imaging devices configured to provide feedback to the robotically-controlled system to control insertion of the access assembly and/or the radiofrequency energy delivery device and/or operation thereof. The one or more imaging devices may be carried by the one or more robotic arms of the robotically-controlled system. In some instances, the robotically-controlled system uses a closed-loop system to control insertion of the access assembly and/or the radiofrequency energy delivery device and/or operation thereof. In some instances, the robotically controlled system changes the configuration of the radiofrequency energy delivery device (e.g., adjusts target treatment location or treatment or operational parameters).

The method may also include displaying on a display a desired trajectory directed to the target treatment site within the vertebral body to a user of the robotically-controlled system. The method may further include receiving an input from the user to modify the desired trajectory directed to the target treatment site. The display may be a display on an augmented reality device (e.g., headset or eyewear, such as augmented reality glasses, or an augmented reality tablet or portal device).

In accordance with several examples, a system for facilitating nerve ablation includes an operator control console comprising a computer-based control system including at least one processor that is configured to execute program instructions stored on a non-transitory computer-readable medium to carry out a nerve ablation procedure to ablate a basivertebral nerve and/or other intraosseous nerve(s) within one or more vertebral bodies using automated robotic surgical arms. The system also includes one or more robotic surgical arms configured to move with six or more degrees of freedom and to support or carry access tools, treatment devices, and/or diagnostic devices. The system further includes a tracking system that can be used to capture a position of at least a portion of a patient, the access tools or treatment devices, and the one or more robotic surgical arms. The tracking system may include one or more imaging devices configured to obtain images of a target treatment site and/or one or more sensors, trackers or markers (e.g., optical sensors, electromagnetic sensors, LIDAR sensors, infrared sensors, ultrasound sensors, force sensors, motion sensors, proximity sensors) to facilitate detection or position tracking and/or registration. The robotic system also includes a display, wherein the computer-based control system is configured to display a desired trajectory directed to a target treatment site within the vertebral body to perform the nerve ablation procedure.

In some examples, the one or more imaging devices are carried by the one or more robotic surgical arms of the system. In some examples, the one or more robotic surgical arms are carried by a mobile cart. The computer-based control system may use a closed-loop system to control insertion of the carry access tools, treatment devices, and/or diagnostic devices. The system may further include a display, wherein the computer-based control system is configured to display a desired trajectory directed to the target treatment site within the vertebral body. In some configurations, the computer-based control system is configured to receive an input from a user to modify the desired trajectory directed to the target treatment site. The computer-based control system may be configured to display actual images and/or graphical representations of the access tools, treatment devices, and/or diagnostic devices on an actual image and/or graphical representation of the target treatment site.

In some examples, the display comprises a see-through display on an augmented reality or a virtual reality device (e.g., headset, goggles, glasses, eyewear, computer tablet or portal) and the robotic system is configured for a user to operate in an augmented reality environment or a virtual reality environment. The operator control console may comprise an augmented reality device.

In some configurations, the robotic system includes a surgical instrument guide configured to be coupled to the one or more robotic surgical arms to facilitate guided insertion of the access tools or treatment devices along the desired trajectory. In some configurations, the computer-based control system uses a closed-loop system to control automated insertion of the access tools or treatment devices. In some configurations, the closed-loop system incorporates feedback based on one or more trained neural networks.

In accordance with several implementations, a robotic-assisted method of ablating a basivertebral nerve and/or other intraosseous nerve within a vertebral body includes positioning, with a robotically-controlled system, a surgical instrument guide along a trajectory directed to a target treatment site within the vertebral body. The method also includes inserting an access assembly through the surgical instrument guide and into the vertebral body. The method further includes inserting a radiofrequency energy delivery device through the access assembly to the target treatment site within the vertebral body and applying power (e.g., a thermal treatment dose) to the target treatment site using the radiofrequency energy delivery device sufficient to ablate the basivertebral nerve and/or other intraosseous nerve.

In some implementations, the robotically-controlled system comprises one or more robotic arms. The one or more robotic arms may have at least six degrees of freedom; however, other degrees of freedom may be used. The robotically-controlled system may include an operator control console comprising at least one processor. The system may also include one or more imaging devices configured to provide feedback to the robotically-controlled system to control insertion of the access assembly and/or the radiofrequency energy delivery device and/or operation thereof. In some implementations, the one or more imaging devices are carried by one or more arms of the robotically-controlled system. In some implementations, the robotically-controlled system uses a closed-loop system to control insertion of the access assembly and/or the radiofrequency energy delivery device and/or operation thereof. The closed-loop system may incorporate feedback based on artificial intelligence algorithms implementing one or more trained neural networks.

The trajectory may be determined automatically by the robotically-controlled system based on (i) pre-operative images of a vertebra corresponding to the vertebral body and surrounding patient anatomy; (ii) a location of the vertebral body; and/or (iii) patient-specific characteristics associated with the vertebral body.

In some configurations, the trajectory is configured to end at a region that includes a basivertebral nerve trunk. The trajectory may include one or more path subcomponents and various subcomponents may be straight or curved.

The pre-operative images may include magnetic resonance images or computed tomography images of a portion of the patient's spine. Intraoperative images (e.g., fluoroscopic images) may additionally or alternatively be used. The location of the vertebral body may include an identification of whether the vertebral body is of a lumbar vertebra or a sacral vertebra and a particular level of the vertebra (e.g., L5, L4, L3, S1, S2). The patient-specific characteristics may include for example, a bone density measurement of one or more vertebral bodies of the patient.

In some implementations, the trajectory is determined by a user (e.g., surgeon or other clinical professional) based on pre-operative images of at least the vertebral body.

In some examples, the method also includes displaying the desired trajectory directed to the target treatment site within the vertebral body to a user of the robotically-controlled system. The method may also include receiving instructions from the user modifying the desired trajectory directed to the target treatment site.

In some examples, the method further includes manually inserting the access assembly through the surgical instrument guide and into the vertebral body and/or manually inserting a radiofrequency energy delivery device through the access assembly to the target treatment site within the vertebral body. The method may include automatically adjusting, via the robotically-controlled system, a position of the surgical instrument guide based on change in position of the vertebral body such that a spatial relationship between the surgical instrument guide and the vertebral body remains substantially unaltered as at least a portion of an operation of ablating a basivertebral nerve and/or other intraosseous nerves is performed.

In accordance with several examples, a system for facilitating nerve ablation includes an operator control console comprising a computer-based control system including at least one processor that is configured to execute program instructions stored on a non-transitory computer-readable medium to carry out a nerve ablation procedure to ablate a basivertebral nerve and/or other intraosseous nerve(s) within one or more vertebral bodies using automated robotic surgical arms. The system also includes a robotic surgical arm configured to move with at least three degrees of freedom and to support a surgical instrument guide. The system further includes a tracking system to capture the position of the robotic surgical arm and an access assembly and/or a radiofrequency energy delivery device configured to be inserted through the surgical instrument guide.

In some configurations, the computer-based control system is configured to provide feedback to a user to control insertion of the access assembly and/or the radiofrequency energy delivery device and/or operation thereof. In some example configurations, the computer-based control system uses a closed-loop system to control insertion of the access assembly and/or the radiofrequency energy delivery device and/or operation thereof. The computer-based control system may be configured to display on a display a desired trajectory directed to a target treatment site within a vertebral body to a user of the system.

In accordance with several implementations, a method of facilitating nerve ablation in a patient includes generating, by a computer system comprising at least one processor, a virtual trajectory, wherein the virtual trajectory is a virtual axis, wherein the virtual axis is a three-dimensional digital representation indicating a position, orientation, or combination thereof, for advancing one or more physical surgical tools or physical surgical instruments towards a treatment site. The method also includes displaying a position, orientation, or combination thereof, using the display, of the virtual surgical axis onto a representation of a portion of the patient anatomy so as to superimpose the virtual trajectory onto patient's anatomy and wherein the display of the position, orientation, or combination thereof, of the virtual axis is configured to be maintained by the computer system in relationship to one or more anatomic structures when the patient moves, wherein the one or more anatomic structures are registered in a coordinate system, wherein the virtual axis is registered in the coordinate system.

In some implementations, the method further includes displaying the virtual axis with a see-through optical head mounted display that is registered in the coordinate system. The method may further include inserting a surgical tool along the virtual trajectory and into a vertebral body of the patient and ablating a basivertebral nerve trunk within the vertebral body of the patient.

In accordance with several implementations, a system for facilitating nerve ablation in a patient includes at least one processor, at least one display, and at least one user interface. The system is configured to generate a virtual trajectory, wherein the virtual trajectory is a virtual axis, wherein the virtual axis is a three-dimensional digital representation indicating desired insertion path of a plurality of instruments towards a target site within a vertebral body, and wherein the target site corresponds to a location of a basivertebral nerve trunk within the vertebral body.

In accordance with several examples, a system for facilitating nerve ablation includes an operator control console comprising a computer-based control system including at least one processor that is configured to execute program instructions stored on a non-transitory computer-readable medium to carry out a nerve ablation procedure to ablate a basivertebral nerve within one or more vertebral bodies using automated robotic surgical arms. The one or more robotic surgical arms are configured to move with six or more degrees of freedom and to support or carry bone access tools (e.g., cannulas, stylets, bone drills, curettes), treatment devices (e.g., radiofrequency probes, microwave ablation catheters, ultrasound probes), and/or diagnostic devices (e.g., cameras, sensors, and/or the like). The system may optionally include one or more imaging devices configured to obtain images of a target treatment site prior to, during, and/or after a treatment procedure.

In accordance with several examples, a method of ablating a basivertebral nerve and/or other intraosseous nerve within a vertebral body includes inserting an access assembly (e.g., bone access introducer cannula and/or stylet) within a vertebral body using a robotically-controlled system. The method also includes inserting an ablation device (e.g., radiofrequency energy delivery device, microwave energy delivery device, laser energy delivery device, ultrasound energy delivery device, thermal energy delivery device, cryoablation device, chemical ablation device, steam or vapor delivery device) through the cannula to a target treatment site within the vertebral body using a robotically-controlled system and providing treatment (e.g., applying power) to the target treatment site using the ablation device sufficient to ablate the basivertebral nerve and/or other intraosseous nerve(s).

In accordance with several implementations, a method of facilitating ablation of a basivertebral nerve within a vertebral body comprising applying radiofrequency energy to a location within the vertebral body according to the following treatment parameters: a frequency between 400 kHz and 600 kHz (e.g., between 400 kHz and 500 kHz, between 450 kHz and 500 kHz, between 470 kHz and 490 kHz, between 500 kHz and 600 kHz, overlapping ranges thereof, or any value within the recited ranges); a target temperature of between 80 degrees Celsius and 90 degrees Celsius (e.g., 80 degrees Celsius, 85 degrees Celsius, 90 degrees Celsius); a temperature ramp of between 0.5 and 3 degrees Celsius per second (e.g., 0.5 degree Celsius per second, 1 degree Celsius per second, 1.5 degrees Celsius per second, 2 degrees Celsius per second, 2.5 degrees Celsius per second, 3 degrees Celsius per second); and an active energy delivery time of between 10 minutes and 20 minutes (e.g., 10 minutes, 12, minutes, 14 minutes, 15 minutes, 16 minutes, 18 minutes, 20 minutes). In some implementations, a target ablation zone has a major diameter along a long axis of between 20 mm and 30 mm and a minor diameter along a short axis of between 5 mm and 15 mm. In accordance with several examples, a robotically-enabled or robotically-controlled surgical system can be configured to position and/or operate the probe to facilitate ablation of the basivertebral nerve within a vertebral body as according to the method described above.

In accordance with several configurations, a kit for facilitating nerve ablation includes one or more biological assays configured to determine at least one biological marker (e.g., cytokine, substance P or other indicator of pain, heat shock protein). The determination includes at least one of a binary detection of a presence of the at least one biological marker, and/or a quantification (e.g., total amount) of the at least one biological marker. The determination may also optionally include an indication of location of any of the at least one biomarker or a location of a highest concentration of the at least one biomarker. In accordance with several configurations, a robotically-enabled or robotically-controlled surgical system is configured to obtain and/or display images, and/or measurements from a patient prior to treatment and after treatment and the presence of one or more biomarkers in the pre-treatment and post-treatment samples may be compared to confirm treatment efficacy. The comparison may involve comparison of levels or activity of the biomarkers within the samples and displaying such results to the user with the robotic surgical system.

The kit may optionally include one or more bone access tools (e.g., stylets, cannulas, curettes, bone drills) configured to access a target nerve to be treated (e.g., basivertebral nerve). The kit may also or alternatively optionally include one or more treatment tools configured to modulate (e.g., ablate, stimulate, denervate, inhibit, necrose, electroporate, molecularly dissociate) the target nerve. The optional treatment tool includes one or a combination of the following: a radiofrequency energy delivery device, a microwave energy delivery device, an ultrasound energy delivery device, a cryomodulation device (e.g., cryoablation device), a laser energy delivery device, and/or a drug eluting device (e.g., chemical or fluid ablation device configured to elute a fluid capable of denervating or ablating a nerve, such as alcohol or phenol). In accordance with several configurations, a robotically-enabled or robotically-controlled surgical system can be configured to position a surgical guide instrument though which one or more bone access tools (e.g., stylets, cannulas, curettes, bone drills) can be inserted to access a target nerve to be treated (e.g., basivertebral nerve.

In accordance with several implementations, a method of detecting and treating back pain of a subject includes obtaining images of a vertebral body of the subject and analyzing the images to determine whether the vertebral body exhibits one or more symptoms associated with a pre-Modic change. The method also includes modulating (e.g., ablating, denervating, stimulating) an intraosseous nerve (e.g., basivertebral nerve) within the vertebral body if it is determined that the vertebral body exhibits one or more symptoms associated with a pre-Modic change. In accordance with several configurations, a robotically-enabled or robotically-controlled surgical system can be configured to control modulation.

The images may be 2D, 3D, or 4D images obtained, for example, using an MRI imaging modality, a CT imaging modality, an X-ray imaging modality, an ultrasound imaging modality, or fluoroscopy. The one or more symptoms associated with a pre-Modic change may comprise characteristics likely to result in Modic changes (e.g., Type 1 Modic changes, Type 2 Modic changes). The one or more symptoms associated with a pre-Modic change may comprise initial indications or precursors of edema or inflammation at a vertebral endplate prior to a formal characterization or diagnosis as a Modic change. The one or more symptoms may include edema, inflammation, and/or tissue change within the vertebral body or along a portion of a vertebral endplate of the vertebral body. Tissue changes may include tissue lesions or changes in tissue type or characteristics of an endplate of the vertebral body and/or tissue lesions or changes in tissue type or characteristics of bone marrow of the vertebral body. The one or more symptoms may include focal defects, erosive defects, rim defects, and corner defects of a vertebral endplate of the vertebral body.

The thermal treatment dose applied may include delivery of one or more of radiofrequency energy, ultrasound energy, microwave energy, and laser energy. Ablating the basivertebral nerve within the vertebral body may comprise applying a thermal treatment dose to a location within the vertebral body of at least 240 cumulative equivalent minutes (“CEM”) using a CEM at 43 degrees Celsius model. In some implementations, the thermal treatment dose is between 200 and 300 CEM (e.g., between 200 and 240 CEM, between 230 CEM and 260 CEM, between 240 CEM and 280 CEM, between 235 CEM and 245 CEM, between 260 CEM and 300 CEM) or greater than a predetermined threshold (e.g., greater than 240 CEM). The target thermal treatment dose may also be based on an Arrhenius model.

In some implementations, ablating the basivertebral nerve within the vertebral body comprises advancing at least a distal end portion of a radiofrequency energy delivery probe comprising two electrodes (e.g., a bipolar probe having an active electrode and a return electrode) to a target treatment location within the vertebral body and applying radiofrequency energy to the location using the energy delivery probe to generate a thermal treatment dose sufficient to modulate (e.g., ablate, denervate, stimulate) the intraosseous nerve (e.g., basivertebral nerve). The radiofrequency energy may have a frequency between 400 kHz and 600 kHz (e.g., between 400 kHz and 500 kHz, between 425 kHz and 475 kHz, between 450 kHz and 500 kHz, between 450 kHz and 550 kHz, between 475 kHz and 500 kHz, between 500 kHz and 600 kHz, overlapping ranges thereof, or any value within the recited ranges). In some instances, the thermal treatment dose is configured to achieve a target temperature of between 70 degrees Celsius and 95 degrees Celsius (e.g., between 70 degrees Celsius and 85 degrees Celsius, between 80 degrees Celsius and 90 degrees Celsius, between 85 degrees Celsius and 95 degrees Celsius, overlapping ranges thereof, or any value within the recited ranges) at the location. The thermal treatment dose may be delivered with a temperature ramp of between 0.1 and 5 degrees Celsius per second (e.g., between 0.5 and 1.5 degrees Celsius per second, between 1 and 2 degrees Celsius per second, between 1.5 and 3 degrees Celsius per second, between 0.5 and 3 degrees Celsius per second, between 1.5 and 5 degrees Celsius per second, overlapping ranges thereof, or any value within the recited ranges. In some implementations, the temperature ramp is greater than 5 degrees Celsius per second. The radiofrequency energy may be applied for an active energy delivery time of between 5 minutes and 30 minutes (e.g., between 5 minutes and 15 minutes, between 10 minutes and 20 minutes, between 15 minutes and 30 minutes, overlapping ranges thereof, or any value within the recited ranges). The thermal treatment dose may form a targeted lesion zone at the target treatment location having a maximum cross-sectional dimension of less than 15 mm. In some implementations, the radiofrequency energy delivery probe is controlled and operated by a surgical robotic system.

Ablating the basivertebral nerve may comprise generating a targeted ablation zone formed by a lesion having a “football” or elliptical profile shape. Ablating the basivertebral nerve may comprise generating a targeted ablation zone having a maximum cross-sectional dimension (e.g., diameter, height, width, length) of less than 15 mm. In some implementations, ablating the basivertebral nerve comprises generating a targeted ablation zone having a maximum cross-sectional dimension (e.g., major diameter) along a long axis of between 20 mm and 30 mm and a maximum cross-sectional dimension (e.g., minor diameter) along a short axis of between 5 mm and 15 mm.

In some implementations, the method is performed without use of any cooling fluid. The method may further include modulating (e.g., ablating, denervating, stimulating) an intraosseous nerve (e.g., basivertebral nerve) within a second vertebral body superior to or inferior to the first vertebral body.

In accordance with several implementations, a method of detecting and treating back pain of a subject includes identifying a candidate vertebral body for treatment based on a determination that the vertebral body exhibits one or more symptoms or defects associated with vertebral endplate degeneration and ablating a basivertebral nerve within the identified candidate vertebral body by applying a thermal treatment dose to a location within the vertebral body of at least 240 cumulative equivalent minutes (“CEM”) using a CEM at 43 degrees Celsius model. The one or more symptoms associated with vertebral endplate degeneration or defects include pre-Modic change characteristics.

In some instances, the determination is based on images of the candidate vertebral body (e.g., MRI images, CT images, X-ray images, fluoroscopic images, ultrasound images). In some instances, the determination is based on obtaining biomarkers from the subject. The biomarkers may be obtained, for example, from one or more blood serum samples (e.g., blood plasma). The biomarkers may be obtained over an extended period of time (e.g., a period of days, weeks, or months) or at a single instance in time. In some embodiments, images of the candidate vertebral body are displayed on a display of a robotic surgical system.

In some implementations, the location of the applied thermal treatment dose is in a posterior half of the vertebral body. The location may include a geometric center of the vertebral body. The location may be at least 5 mm (e.g., at least 1 cm) from a posterior border (e.g., posterior cortical aspect) of the vertebral body.

In some implementations, the method includes advancing at least a distal end portion of a bipolar radiofrequency energy delivery probe having two electrodes to the location. The method may further include forming a passageway through a pedicle and into the vertebral body, then advancing at least the distal end portion of the bipolar radiofrequency energy delivery probe along the passageway to the location, and then applying the thermal treatment dose to the location using the bipolar radiofrequency energy delivery probe. In some implementations, the method includes advancing the bipolar radiofrequency energy delivery probe through a surgical guide instrument that is positioned by a robotically controlled system. In some implementations, movement and/or position of the surgical guide instrument and/or the energy delivery probe are coupled to robotic arms of the robotic surgical system.

In some implementations, the method further includes applying radiofrequency energy to a second location within a second vertebral body. The second vertebral body may be of a vertebra of a different vertebral level than the first vertebral body. The second vertebral body may be of a vertebra adjacent to the first vertebral body.

In accordance with several configurations, an introducer system adapted to facilitate percutaneous access to a target treatment location within bone (e.g., a vertebral body) includes an introducer cannula comprising a proximal handle and a distal elongate hypotube extending from the proximal handle. The system further includes an introducer stylet comprising a proximal handle and a distal elongate shaft extending from the proximal handle. The proximal handle of the introducer includes a central opening in its upper surface that is coupled to a lumen of the distal elongate hypotube to facilitate insertion of the introducer stylet into the central opening and into the distal elongate hypotube of the introducer cannula. The proximal handle of the introducer cannula includes one or more slots configured to receive at least a portion of the proximal handle of the introducer stylet so as to facilitate engagement and alignment between the introducer stylet and the introducer cannula. The proximal handle of the introducer stylet includes an anti-rotation tab configured to be received within one of the one or more slots so as to prevent rotation of the introducer stylet within the introducer cannula. A distal end of the distal elongate shaft of the introducer stylet includes a distal cutting tip and a scalloped section proximal to the distal cutting tip so as to provide gaps between an outer diameter of the distal end of the distal elongate shaft and the inner diameter of the introducer cannula. In accordance with several configurations, a robotically-enabled or robotically-controlled surgical system can be configured to introduce the introducer system through skin adjacent the vertebral body and into a pedicle connected to the vertebral body. In accordance with several configurations, a robotically-enabled or robotically-controlled surgical system can be configured to position a surgical guide instrument such that the introducer system can be inserted through the surgical guide instrument through skin adjacent the vertebral body and into a pedicle connected to the vertebral body.

In some arrangements, the proximal handle of the introducer stylet further includes a press button that, when pressed: (a) disengages the anti-rotation tab and allows for rotation of the introducer stylet within the introducer stylet, and (b) allows for removal of the introducer stylet from the introducer cannula. The proximal handle of the introducer stylet may include a ramp configured to provide a mechanical assist for removal of the introducer stylet from the introducer cannula. The proximal handle of the introducer cannula may comprise a T-shaped, or smokestack shaped, design.

The introducer system may further include a curved cannula assembly. The curved cannula assembly may include a cannula comprising a proximal handle with a curved insertion slot and a distal polymeric tube. The distal polymeric tube may include a curved distal end portion having a preformed curvature but configured to bend when placed under constraint (e.g., constraint by insertion through a straight introducer cannula). The curved cannula assembly may further include a stylet comprising a proximal handle and a distal elongate shaft. The distal elongate shaft includes a curved distal end portion having a preformed curvature but configured to bend when placed under constraint (e.g., constraint by insertion through a cannula or bone tissue) and a distal channeling tip. A length of the curved distal end portion of the distal elongate shaft proximal to the distal channeling tip (e.g., a springboard or platform portion) may comprise a cross-section circumference profile that is less than a full cross-section circumference profile (e.g., cross-section circumference profile of neighboring or adjacent portions of the distal elongate shaft or of the distal channeling tip), such that there is a larger gap between an outer cross-sectional dimension of the curved distal end portion of the distal elongate shaft and the inner diameter of the curved distal end portion of the cannula along the length of the curved distal end portion of the distal elongate shaft proximal to the distal channeling tip. The less than full cross-section circumference profile may comprise a “D” shape. The overall cross-section circumference profile may thus be asymmetric (e.g., not uniform or constant along its entire length).

The proximal handle of the stylet may include an actuation mechanism or means for actuating (e.g., bail actuator, threaded knob or screw actuation mechanism, sliding actuator, pullwire actuator, lever, hydraulic actuator, pneumatic actuator, electrical actuator, push button actuator, mechanical linear actuator). The actuation mechanism or means for actuating is adapted to cause axial movement (e.g., proximal movement upon actuation) of the distal channeling tip of the distal elongate shaft of the stylet with respect to the cannula so as to facilitate insertion of the curved cannula assembly through the introducer cannula and withdrawal of the stylet of the curved cannula assembly from the cannula of the curved cannula assembly after formation of a curved path within the bone.

In accordance with several implementations, a method of accessing a target treatment location within a vertebral body identified as having hard bone includes advancing an introducer assembly through skin adjacent the vertebral body and into a pedicle connected to the vertebral body, the introducer assembly including an introducer stylet inserted within an introducer cannula with a distal cutting tip of the introducer stylet extending out of the introducer cannula. The method further includes removing the introducer stylet from the introducer cannula while leaving the introducer cannula in place. The method also includes inserting an introducer drill through and beyond the introducer cannula and through the pedicle and into cancellous bone of the vertebral body. Inserting the introducer drill includes rotating the introducer drill. The introducer drill includes a fluted distal portion and a distal drill tip. The drill flutes of the fluted distal portion taper away from the distal drill tip so as to facilitate improved bone chip packing within an open volume defined by the drill flutes as bone chips are generated by operation of the introducer drill. In accordance with several configurations, a robotically-enabled or robotically-controlled surgical system can be configured to introduce an introducer assembly through skin adjacent the vertebral body and into a pedicle connected to the vertebral body. In accordance with several configurations, a robotically-enabled or robotically-controlled surgical system can be configured to position a surgical guide instrument such that an introducer assembly can be inserted through the surgical guide instrument through skin adjacent the vertebral body and into a pedicle connected to the vertebral body.

In accordance with several implementations, inserting the introducer drill may involve not malleting on the introducer drill. In some implementations, inserting the introducer drill does include malleting on a proximal handle of the introducer drill. The method may further include removing the introducer drill from the introducer cannula. The method may also include inserting a curved cannula assembly into a curved slot of a proximal handle of the introducer cannula. The curved cannula assembly may include a second cannula including a proximal handle with a curved insertion slot and a distal polymeric tube, wherein the distal polymeric tube includes a curved distal end portion having a preformed curvature but configured to bend when placed under constraint. The curved cannula assembly may also include a second stylet including a proximal handle and a distal elongate shaft. The distal elongate shaft of the second stylet includes a curved distal end portion having a preformed curvature but configured to bend when placed under constraint and a distal channeling tip. A length of the curved distal end portion of the distal elongate shaft proximal to the distal channeling tip may comprise a cross-section circumference profile that is less than a full cross-section circumference profile such that there is a larger gap between an outer cross-sectional dimension of the curved distal end portion of the distal elongate shaft and the inner diameter of the curved distal end portion of the second cannula along the length of the curved distal end portion of the distal elongate shaft proximal to the distal channeling tip. In accordance with several configurations, a robotically-enabled or robotically-controlled surgical system can be configured to remove and insert introducer drill and/or curved cannula assembly.

In some implementations, the method further includes removing the second stylet from the second cannula. The method may also include inserting a third stylet into a slot of the proximal handle of the second cannula and beyond an open distal tip of the second cannula, wherein the third stylet is configured to form a straight path (e.g., beyond a curved path formed by the curved cannula assembly) starting from the open distal tip of the second cannula toward the target treatment location, and removing the third stylet from the second cannula after formation of the straight path. The method may include inserting a treatment device into the slot of the proximal handle of the second cannula and beyond the open distal tip of the second cannula to the target treatment location and performing therapy at the target treatment location using the treatment device. The therapy may include ablating at least 75% of the branches of a basivertebral nerve within the bone (e.g., vertebral body). In accordance with several configurations, a robotically-enabled or robotically-controlled surgical system can be configured to remove and insert the instrument described above.

Several embodiments of the invention have one or more of the following advantages: (i) increased treatment accuracy; (ii) increased efficacy and enhanced safety; (iii) increased efficiency; (iv) increased precision; (v) synergistic results; (vi) “one-and-done” procedure that does not require further surgical intervention; (vii) treatment of chronic low back pain; (viii) prevention of pain due to early detection of factors likely to cause pain in the future; (ix) ease of use (e.g., due to reduced friction or force) and/or (x) robotic control or guidance to facilitate access or navigation.

For purposes of summarizing the disclosure, certain aspects, advantages, and novel features of embodiments of the disclosure have been described herein. It is to be understood that not necessarily all such advantages may be achieved in accordance with any particular embodiment of the disclosure provided herein. Thus, the embodiments disclosed herein may be embodied or carried out in a manner that achieves or optimizes one advantage or group of advantages as taught or suggested herein without necessarily achieving other advantages as may be taught or suggested herein.

The methods summarized above and set forth in further detail below describe certain actions taken by a practitioner; however, it should be understood that they can also include the instruction of those actions by another party. Thus, actions such as “applying thermal energy” include “instructing the applying of thermal energy.” Further aspects of embodiments of the disclosure will be discussed in the following portions of the specification. With respect to the drawings, elements from one figure may be combined with elements from the other figures.

### BRIEF DESCRIPTION OF THE DRAWINGS

Several embodiments of the disclosure will be more fully understood by reference to the following drawings which are for illustrative purposes only:

FIG. 1 illustrates various vertebral levels and vertebrae that may be treated by the systems and methods described herein.

FIG. 2 illustrates pelvic bones of a human to illustrate potential methods of accessing certain vertebral bodies.

FIG. 3 illustrates an example kit or system of access tools configured to access a vertebral body.

FIGS. 3A-3H illustrate various steps of a method of accessing and treating tissue within a vertebral body using one or more of the access tools of the kit or system of FIG. 3.

FIG. 4 illustrates a schematic block diagram of a robotically-enabled system.

FIG. 5 illustrates an example of a robotically-enabled system positioned within a surgery room.

FIG. 5A illustrates a lateral view of a spine which can be displayed on a display of a robotically-enabled system.

FIG. 5B illustrates a posterior view of a spine which can be displayed on a display of a robotically-enabled system.

FIG. 5C illustrates an axial view of a spine which can be displayed on a display of a robotically-enabled system.

FIG. 6 illustrates an example of a robotic arm of the robotically-enabled system of FIG. 5.

FIG. 7 illustrates an example of a surgical guide of the robotic arm of FIG. 6.

FIG. 8 illustrates a surgical tool being inserted through the surgical guide of FIG. 7.

FIG. 9 illustrates an example of an introducer cannula that can be inserted through the surgical guide of FIG. 7.

### DETAILED DESCRIPTION

Several implementations described herein are directed to systems and methods for modulating nerves within or adjacent (e.g., surrounding) bone using a robotic system. Robotic systems may be integrated with navigation systems. The robotic system or navigation system integrated with the robotic system may incorporate augmented reality systems and devices that incorporate real-time navigation images from intraoperative imaging (e.g., fluoroscopic or CT guidance imaging) with a 3D-reconstructed model (e.g., 3D model of a portion of a spine) in the surgeon's field of view. The 3D-reconstructed model may be created from pre-operative images (e.g., MRI images or CT images) of the particular patient. Virtual reality systems and devices may be incorporated to facilitate training of orthopedic surgeons on performing various spine surgeries or spine treatment procedures (e.g., spinal neuromodulation procedures such as ablation of intraosseous nerves within one or more vertebral bodies or ablation of nerves innervating one or more vertebral endplates and/or intervertebral discs). In some implementations, an intraosseous nerve (e.g., basivertebral nerve or other nerves branching from a sinuvertebral nerve) within a bone (e.g., vertebral body) of the spine is modulated for treatment, or prevention of, chronic back pain. The nerve(s) may be one or more nerves that innervate a vertebral endplate. The vertebral body may be located in any level of the vertebral column (e.g., cervical, thoracic, lumbar and/or sacral).

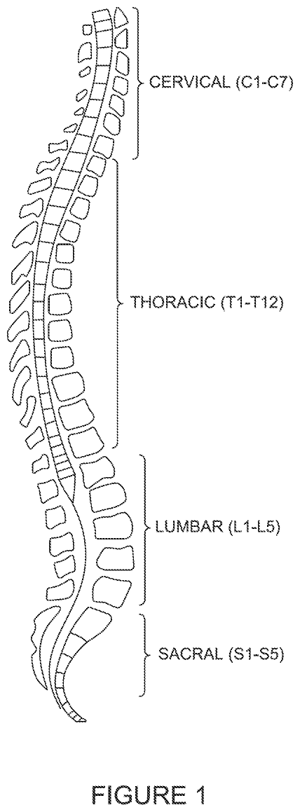

FIG. 1 schematically illustrates a vertebral column and the various vertebral segments or levels. Multiple vertebral bodies may be treated in a single visit or procedure (simultaneously or sequentially). The multiple vertebral bodies may be located in a single spine segment (e.g., two adjacent vertebral bodies in the sacral spine segment (e.g., S1 and S2) or lumbar spine segment (e.g., L3, L4 and/or L5) or thoracic spine segment or cervical spine segment) or in different spine segments (e.g., an L5 vertebra in the lumbar spine segment and an S1 vertebra in the sacral spine segment). Intraosseous nerves within bones other than vertebral bodies may also be modulated. For example, nerves within a humerus, radius, femur, tibia, calcaneus, tarsal bones, hips, knees, and/or phalanges may be modulated.

In some implementations, the one or more nerves being modulated are extraosseous nerves located outside the vertebral body or other bone (e.g., at locations before the nerves enter into, or after they exit from, a foramen of the bone). Other tissue in addition to, or alternative to, nerves may also be treated or otherwise affected (e.g., tumors or other cancerous tissue or fractured bones). Portions of nerves within or on one or more vertebral endplates or intervertebral discs between adjacent vertebral bodies may be modulated.

The modulation of nerves or other tissue may be performed to treat one or more indications, including but not limited to chronic low back pain, upper back pain, acute back pain, joint pain, tumors in the bone, and/or bone fractures. The modulation of nerves may also be performed in conjunction with bone fusion or arthrodesis procedures so as to provide synergistic effects or complete all-in-one, “one-and-done” treatment that will not require further surgical or minimally invasive interventions.

In some implementations, fractures within the bone may be treated in addition to denervation treatment and/or ablation of tumors by applying heat or energy and/or delivering agents or bone filler material to the bone. For example, bone morphogenetic proteins and/or bone cement may be delivered in conjunction with vertebroplasty or other procedures to treat fractures or promote bone growth or bone healing. In some implementations, energy is applied and then agents and/or bone filler material is delivered in a combined procedure. In some aspects, vertebral compression fractures (which may be caused by osteoporosis or cancer) are treated in conjunction with energy delivery to modulate nerves and/or cancerous tissue to treat back pain.

In accordance with several implementations, the systems and methods of treating back pain or facilitating neuromodulation of intraosseous nerves (e.g., robotic systems and methods) described herein can be performed without surgical resection, without general anesthesia, without cooling (e.g., without cooling fluid), and/or with virtually no blood loss. In various implementations, the systems and methods described herein are used to deliver non-implantable bone access tools and treatment devices. In some implementations, the systems and methods described herein allow for decreased radiation exposure and decreased operational procedure time. In some implementations, the systems and methods of treating back pain or facilitating neuromodulation of intraosseous nerves described herein facilitate easy retreatment if necessary. In accordance with several implementations, successful treatment can be performed in challenging or difficult-to-access locations and access can be varied depending on bone structure (e.g., different bone density) or differing bone anatomy (e.g., different vertebral levels). One or more of these advantages also apply to treatment of tissue outside of the spine (e.g., other orthopedic applications or other tissue).

### Access to the Vertebral Body

Methods of Access

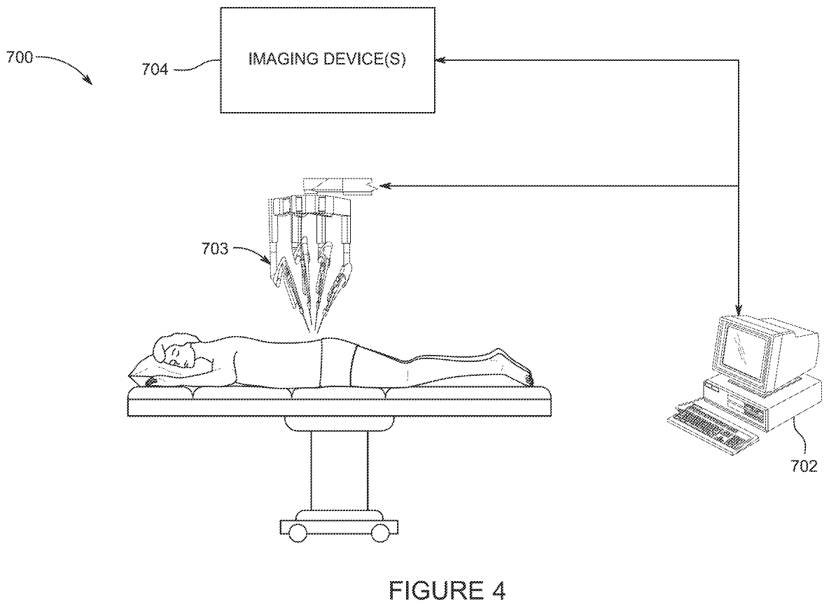

Various methods of access may be used to access a vertebral body or other bone. In some implementations, the vertebral body is accessed transpedicularly (through one or both pedicles). In other implementations, the vertebral body is accessed extrapedicularly (e.g., without traversing through a pedicle). In some implementations, the vertebral body is accessed using an extreme lateral approach or a transforaminal approach, such as used in XLIF and TLIF interbody fusion procedures. In some implementations, an anterior approach is used to access the vertebral body. Access may be assisted using a robotic system, such as the robotic systems described in connection with FIGS. 4-9.

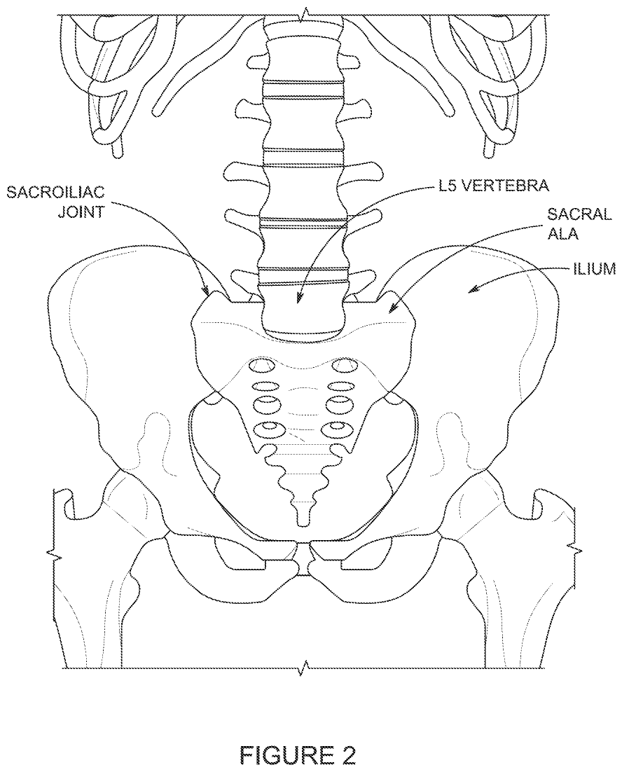

Certain vertebrae in the sacral or lumbar levels (e.g., S1 vertebra, L5 vertebra) may also be accessed generally posterolaterally using a trans-ilium approach (e.g., an approach through an ilium bone). With reference to FIG. 2, an access hole may be formed through the ilium at a location designed to facilitate access to the vertebral body or vertebral bodies in the sacral or lumbar region. For example, access tools (e.g., an introducer assembly including a cannula/stylet combination) may be delivered through an ilium and/or sacroiliac joint or sacral ala into an S1 vertebra under image guidance (e.g., CT image guidance and/or fluoroscopy) and/or using stereotactic or robotic-assisted surgical and/or navigation systems, such as the robotic systems described in connection with FIGS. 4-9. A treatment device could then be inserted through an introducer and/or other access cannula of the access tools to a target treatment location within a sacral or lumbar vertebra. A trans-ilium approach may advantageously increase the ability of the clinician to access the target treatment location in a particular portion or region of the vertebral body (e.g., posterior portion or region) that is not capable of being adequately accessed using a transpedicular approach. In some implementations, the vertebral body may be accessed directly through the cerebrospinal fluid and through the dura into a posterior region of the vertebral body.

In some implementations, the vertebral body may be accessed transforaminally through a basivertebral foramen using stereotactic or robotic-assisted surgical and/or navigation systems, such as the robotic systems described in connection with FIGS. 4-9. Transforaminal access via the spinal canal may involve insertion of a “nerve finder” or nerve locator device and/or imaging/diagnostic tool to avoid damaging spinal cord nerves upon entry by the access tools or treatment devices. The nerve locator device may comprise a hand-held stimulation system such as the Checkpoint Stimulator and Locator provided by Checkpoint Surgical® or the EZstim® peripheral nerve stimulator/nerve locators provided by Avanos Medical, Inc. The nerve finder or nerve locator device could advantageously identify sensitive nerves that should be avoided by the access tools so as not to risk paralysis or spinal cord injury upon accessing the target treatment site. The nerve locator device may be configured to apply stimulation signals between two points or locations and then assess response to determine presence of nerves in the area between the two points or locations. The nerve locator device may include a bipolar pair of stimulation electrodes or monopolar electrodes. In some implementations, the nerve locator features may be implemented on the access tools or treatment devices themselves as opposed to a separate stand-alone device.

Access Tools and Treatment Devices

Access tools (e.g., bone access tools) may include an introducer assembly including an outer cannula and a sharpened stylet, an inner cannula configured to be introduced through the outer cannula, and/or one or more additional stylets, curettes, or drills to facilitate access to an intraosseous location within a vertebral body or other bone. The access tools (e.g., outer cannula, inner cannula, stylets, curettes, drills) may have pre-curved distal end portions or may be actively steerable or curveable. Any of the access tools may have beveled or otherwise sharp tips or they may have blunt or rounded, atraumatic distal tips. Curved drills may be used to facilitate formation of curved access paths within bone. Any of the access tools may be advanced over a guidewire in some implementations.

The access tools may be formed of a variety of flexible materials (e.g., ethylene vinyl acetate, polyethylene, polyethylene-based polyolefin elastomers, polyetheretherketone, polypropylene, polypropylene-based elastomers, styrene butadiene copolymers, thermoplastic polyester elastomers, thermoplastic polyurethane elastomers, thermoplastic vulcanizate polymers, metallic alloy materials such as nitinol, and/or the like). Combinations of two or more of these materials may also be used. The access tools may include chevron or other shape designs or patterns or slits along the distal end portions to increase flexibility or bendability. Any of the access tools may be manually or automatically rotated (e.g., using a robotic control system such as described in connection with FIGS. 4-9) to facilitate a desired trajectory.

In some implementations, an outer cannula assembly (e.g., introducer assembly) includes a straight outer cannula and a straight stylet configured to be received within the outer cannula. The outer cannula assembly may be inserted first through an incision in a skin of the patient to penetrate an outer cortical shell of a bone and provide a conduit for further access tools to the inner cancellous bone. An inner cannula assembly may include a cannula having a pre-curved or steerable distal end portion and a stylet having a corresponding pre-curved or steerable distal end portion. Multiple stylets having distal end portions with different curvatures may be provided in a kit and selected from by a clinician. The inner cannula assembly may alternatively be configured to remain straight and non-curved.

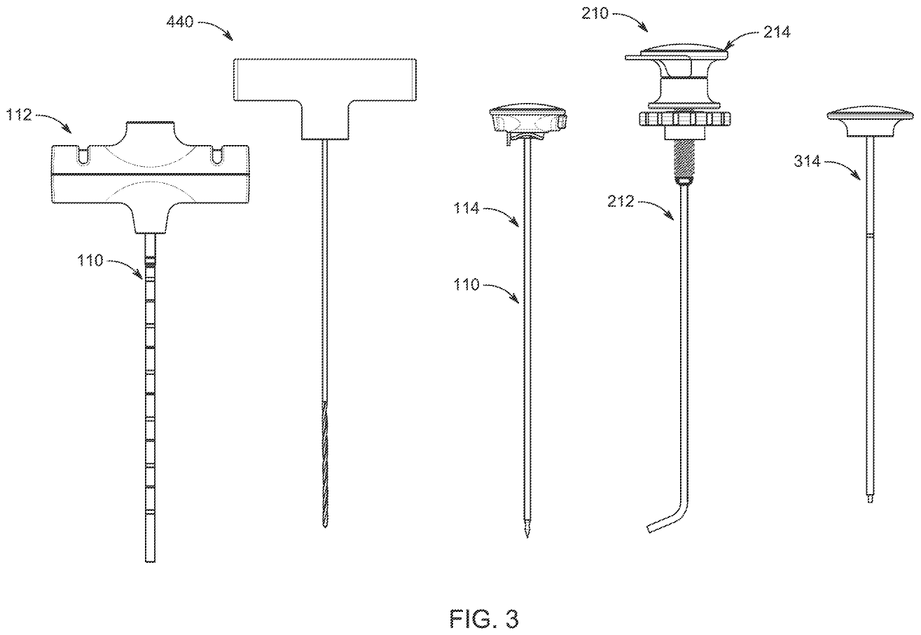

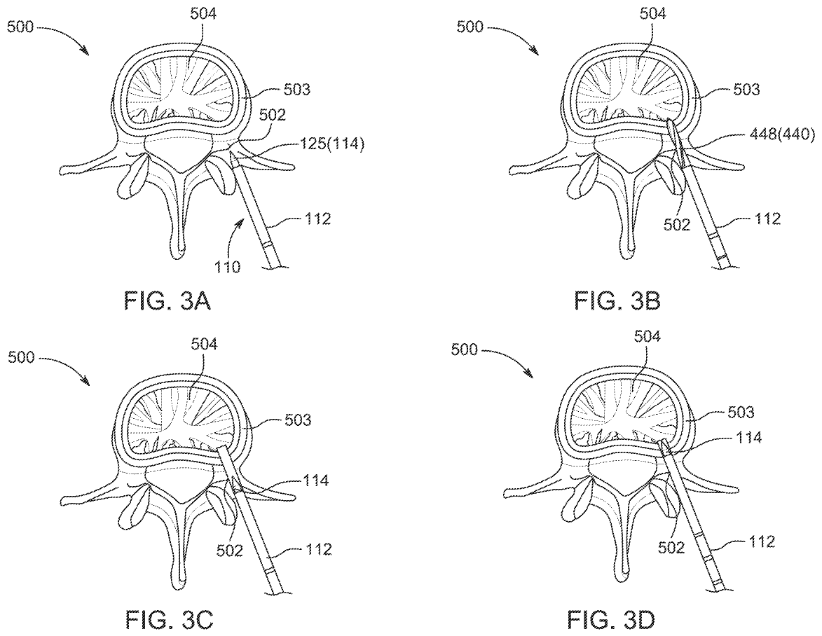

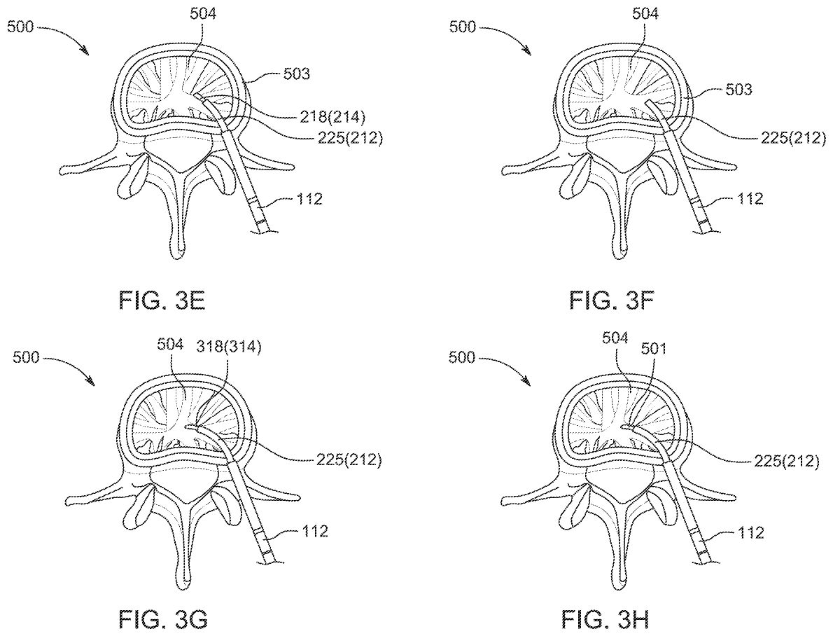

With reference to FIG. 3, in one implementation, a kit or system of access tools includes an introducer assembly 110 comprised of an introducer cannula 112 and an introducer stylet 114, a curved cannula assembly 210 comprised of a curved cannula 212 and a J-stylet 214, and a straight stylet 314. The introducer stylet 114 may be bevel tipped, trocar tipped, and/or diamond tipped. The introducer stylet 114 is configured to be received in a lumen of the introducer cannula 112 in a manner such that a distal tip of the introducer stylet 114 protrudes from an open distal tip of the introducer cannula 112, thereby forming the introducer assembly 110 in combination. The J-stylet 214 is configured to be received in a lumen of the curved cannula 212 in a manner such that a distal tip of the J-stylet 214 protrudes from an open distal tip of the curved cannula 212, thereby forming the curved cannula assembly 210 in combination. The curved cannula 212 and the J-stylet 214 may each comprise a straight proximal main body portion and a curved distal end portion. The curves of the curved distal end portions of the curved cannula 212 and the J-stylet 214 may correspond to each other. The straight stylet 314 is a flexible channeling stylet configured to be delivered through the curved cannula 212 and then to form and maintain a straight or generally straight path upon exiting the open distal tip of the curved cannula 212. Some of the tools may be optional and not used in various implementations.

The access tools may be provided as a kit that may optionally additionally include one or more additional introducer cannulas, one or more additional introducer stylets (e.g., with different tips, such as one with a bevel tip and one with a diamond or trocar tip), one or two or more than two additional curved cannulas (e.g., having a curved distal end portion of a different curvature than a first curved cannula), an additional J-stylet (e.g., having a different curvature or different design configured to access hard bone), an introducer drill, and/or an additional straight stylet (e.g., having a different length than the first straight stylet.

In some arrangements, the access tools (e.g., kit) may be specifically designed and adapted to facilitate access to hard, non-osteoporotic bone (e.g., bone surrounding or within a vertebral body, such as a cervical vertebra, a thoracic vertebra, a lumbar vertebra, or a sacral vertebra). Hard bone may be determined based on bone mass density testing, compressive strength determinations, compressive modulus determinations, imaging modalities, or based on tactile feel by the operator as access instruments are being advanced. In some implementations, hard bone may be determined as bone having a bone mineral density score within a standard deviation of a normal healthy young adult (e.g., a T score greater than or equal to −1). In some implementations, hard bone may be identified as bone having a compressive strength of greater than 4 MPa and/or a compressive modulus of greater than 80 MPa for cancellous bone and greater than 5.5 MPa and/or a compressive modulus of greater than 170 MPa for cortical bone. Some kits may include at least two of every access instrument. Some kits may include optional add-on components or accessory kit modules for accessing hard bone (e.g., the introducer drill 440 and J-stylet 214 specially configured to access hard bone). Some kits may include optional additional access tool components or accessory kit modules adapted to access one or more additional vertebrae in the same spinal segment or in different spinal segments. The kit may also include one or more (e.g., at least two) treatment devices (such as radiofrequency energy delivery probes). Each of the access tools may include one or multiple trackers or sensors (e.g., optical sensors, electromagnetic sensors, ultrasound sensors, force sensors, motion sensors, proximity sensors) to facilitate registration and tracking by a navigation system of the robotic system, such as described further herein.

FIGS. 3A-3H illustrate an example of steps of a method of using the access tools to facilitate access to a location within a vertebral body 500 for treatment (e.g., modulation of intraosseous nerves, such as a basivertebral nerve, bone cement delivery for treatment of vertebral fractures, and/or ablation of bone tumors). One, some, or all of the steps of the method may be performed or facilitated by a robotic system, such as the robotic systems 700,800 described and illustrated in connection with FIGS. 4-9. The robotic system may be configured to operate in a manual mode in which an operator applies force (e.g., malleting or manually pushing, pulling, or rotating) to one or more surgical tools (e.g., access tools, treatment devices or diagnostic devices), a semi-automated mode in which the operator controls one or more robotic arms via a joystick or handheld controller (e.g., in an augmented reality environment using an augmented reality system including a headset and one or more handheld joysticks or other input devices), and/or a fully-automated mode in which the one or more robotic arms are automatically controlled upon execution of a computer program or algorithm. Input from an operator may also be received by voice commands using voice recognition software.

With reference to FIG. 3A, the distal portion of the introducer assembly 110 (including the distal tip 125 of the introducer stylet 114 and the distal tip of the introducer cannula 112) are inserted through a pedicle 502 adjacent the vertebral body 500 after insertion and aligned engagement of the introducer stylet 114 within the introducer cannula 112. The introducer assembly 110 may be coupled to a robotic arm of a robotic system (e.g., system 700, 800) or inserted through a guide positioned by the robotic system.

In accordance with several implementations, the method may optionally include removing the introducer stylet after initial penetration into the pedicle 502 (for example, if the operator determines that the density of the bone is going to be sufficiently dense or hard that additional steps and/or tools will be needed to obtain a desired curved trajectory to access a posterior portion (e.g., posterior half) of the vertebral body 500. With reference to FIG. 3B, the method may optionally include inserting (e.g., via coupling to a robotic arm of a robotic system) an introducer drill 440 into and through the introducer cannula 112 to complete the traversal of the pedicle 502 and penetration through a cortical bone region 503 of the vertebral body 500 until a cancellous bone region 504 of the vertebral body 500 is reached. A distal drill tip 448 of the introducer drill 440 may be advanced into the cancellous bone region 504 (especially if the cancellous bone region 504 is determined to be sufficiently hard or dense) or the advancement may stop at the border between the cortical bone region 503 and the cancellous bone region 504. This step may involve both rotating the introducer drill 440 and advancing of the introducer drill 440 or simply rotating the introducer drill 440. With reference to FIG. 3C, the introducer drill 440 may be removed and the introducer stylet 114 may be re-inserted within the introducer cannula 112. With reference to FIG. 3D, the introducer assembly 110 (e.g., via coupling to a robotic arm of a robotic system) may then be advanced so as to advance the distal tip 122 of the introducer cannula 112 to the entry site into (or within) the cancellous bone region 504 of the vertebral body 500. The introducer stylet 114 may then be removed from the introducer cannula 112.

The curved cannula assembly 210 may then be inserted within the introducer cannula 112. With reference to FIG. 3E, the collective distal end portions of the curved cannula assembly 210 can be advanced together (e.g., in an automated manner via coupling to a robotic arm of a robotic system or via manual malleting on a proximal handle of the J-stylet 214) out of the distal tip of the introducer cannula 112 and along a curved path within the cancellous bone region 504. The curved distal end portion of the J-stylet 214 is configured to always remain within the curved cannula 212 during operation such that only the distal channeling tip 218 or a portion of the distal channeling tip 218 extends out of the curved distal end portion 225 of the curved cannula 212. With reference to FIG. 3F, the J-stylet 214 may then be removed from the curved cannula 212, with the curved cannula 212 remaining in position. In accordance with several implementations, the path formed by the prior instruments may advantageously allow the curved cannula assembly 210 to have a head start and begin curving immediately upon exiting the open distal tip of the introducer cannula 112.

With reference to FIG. 3G, if a further straight path beyond the curved path is desired to reach a target treatment location, the straight stylet 314 may be inserted through the curved cannula 212 (e.g., via coupling to a robotic arm of a robotic system) such that the distal channeling tip 318 of the straight stylet 314 extends beyond the open distal tip of the curved cannula 212 and along a straight path toward the target treatment location (e.g., a basivertebral nerve trunk or basivertebral foramen). In some implementations, the straight stylet 314 may not be needed and this step may be skipped.

With reference to FIG. 3H, a treatment device 501 (e.g., a flexible bipolar radiofrequency probe) may be inserted (e.g., via coupling to a robotic arm of a robotic system) through the curved cannula 212 (after removal of the straight stylet 314 if used) and advanced out of the open distal tip of the curved cannula 212 to the target treatment location. The treatment device 501 may then perform the desired treatment. For example, if the treatment device 501 is a radiofrequency probe, the treatment device 501 may be activated to ablate intraosseous nerves (e.g., a basivertebral nerve) or a tumor within the vertebral body 500. Bone cement or other agent, or a diagnostic device (such as a nerve stimulation device or an imaging device to confirm ablation of a nerve) may optionally be delivered through the curved cannula 212 after the treatment device 501 is removed from the curved cannula 212.

At certain levels of the spine (e.g., sacral and lumbar levels) and for certain patient spinal anatomies that require a steeper curve to access a desired target treatment location within the vertebral body, a combination curette/curved introducer may first be inserted (e.g., via coupling to a robotic arm of a robotic system) to start a curved trajectory (e.g., create an initial curve or shelf) into the vertebra. The curette may have a pre-curved distal end portion or be configured such that the distal end portion can be controllably articulated or curved (e.g., manually by a pull wire or rotation of a handle member coupled to one or more pull wires coupled to the distal end portion or automatically by a robotic or artificial intelligence driven navigation system). The combination curette/curved introducer may then be removed and the outer straight cannula and inner curved cannula/curved stylet assembly may then be inserted to continue the curve toward the target treatment location.

In accordance with several implementations, any of the access tools (e.g., cannula or stylet) or treatment devices may comprise a rheological and/or magnetizable material (e.g., magnetorheological fluid) along a distal end portion of the access tool that is configured to be curved in situ after insertion to a desired location within bone (e.g., vertebra). A magnetic field may be applied to the distal end portion of the access tool and/or treatment device with the magnetizable fluid or other material and adjusted or varied using one or more permanent magnets or electromagnets to cause the distal end portion of the access tool and/or treatment device to curve toward the magnetic field. In some implementations, a treatment probe may include a magnetic wire along a portion of its length (e.g., a distal end portion). Voltage applied to the magnetic wire may be increased or decreased to increase or decrease a curve of the magnetic wire. These implementations may advantageously facilitate controlled steering without manual pull wires or other mechanical mechanisms. The voltage may be applied by instruments controlled and manipulated by an automated robotic control system, such as the robotic system described in connection with FIGS. 4-9.

The treatment devices (e.g., treatment probes) may be any device capable of modulating tissue (e.g., nerves, tumors, bone tissue). Any energy delivery device capable of delivering energy can be used (e.g., RF energy delivery devices, microwave energy delivery devices, laser devices, infrared energy devices, other electromagnetic energy delivery devices, ultrasound energy delivery devices, and the like). The treatment device 501 may be an RF energy delivery device. The RF energy delivery device may include a bipolar pair of electrodes at a distal end portion of the device. The bipolar pair of electrodes may include an active tip electrode and a return ring electrode spaced apart from the active tip electrode. The RF energy delivery device may include one or more temperature sensors (e.g., thermocouples, thermistors) positioned on an external surface of, or embedded within, a shaft of the energy delivery device. The RF energy delivery device may not employ internally circulating cooling, in accordance with several implementations.

In accordance with several implementations, thermal energy may be applied within a cancellous bone portion (e.g., by one or more radiofrequency (RF) energy delivery instruments coupled to one or more RF generators) of a vertebral body. The thermal energy may be conducted by heat transfer to the surrounding cancellous bone, thereby heating up the cancellous bone portion. In accordance with several implementations, the thermal energy is applied within a specific frequency range and having a sufficient temperature and over a sufficient duration of time to heat the cancellous bone such that the basivertebral nerve extending through the cancellous bone of the vertebral body is modulated. In several implementations, modulation comprises permanent ablation or denervation or cellular poration (e.g., electroporation). In some implementations, modulation comprises temporary denervation or inhibition. In some implementations, modulation comprises stimulation or denervation without necrosis of tissue.

For thermal energy, temperatures of the thermal energy may range from about 70 to about 115 degrees Celsius (e.g., from about 70 to about 90 degrees Celsius, from about 75 to about 90 degrees Celsius, from about 83 to about 87 degrees Celsius, from about 80 to about 100 degrees Celsius, from about 85 to about 95 degrees Celsius, from about 90 to about 110 degrees Celsius, from about 95 to about 115 degrees Celsius, or overlapping ranges thereof). The temperature ramp may range from 0.1-5 degrees Celsius/second (e.g., 0.1-1.0 degrees Celsius/second, 0.25 to 2.5 degrees Celsius/second, 0.5-2.0 degrees Celsius/second, 1.0-3.0 degrees Celsius/second, 1.5-4.0 degree Celsius/second, 2.0-5.0 degrees Celsius/second). The time of treatment may range from about 10 seconds to about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy may be delivered as an alternative to or in sequence with continuous energy. For radiofrequency energy, the energy applied may range from 350 kHz to 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz±5 kHz, 475 kHz±5 kHz, 487 kHz±5 kHz). A power of the radiofrequency energy may range from 5 W to 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges). In accordance with several implementations, a thermal treatment dose (e.g., using a cumulative equivalent minutes (CEM) 43 degrees Celsius thermal dose calculation metric model) is between 200 and 300 CEM (e.g., between 200 and 240 CEM, between 230 CEM and 260 CEM, between 240 CEM and 280 CEM, between 235 CEM and 245 CEM, between 260 CEM and 300 CEM) or greater than a predetermined threshold (e.g., greater than 240 CEM). The CEM number may represent an average thermal cumulative dose value at a target treatment region or location and may represent a number that expresses a desired dose for a specific biological end point. An Arrhenius model may alternatively be used to assess and quantify the desired thermal dose. Thermal damage may occur through necrosis or apoptosis.

Cooling may optionally be provided to prevent surrounding tissues from being heated during the nerve modulation procedure. The cooling fluid may be internally circulated through the delivery device from and to a fluid reservoir in a closed circuit manner (e.g., using an inflow lumen and an outflow lumen). The cooling fluid may comprise pure water or a saline solution having a temperature sufficient to cool electrodes (e.g., 2-10 degrees Celsius, 5-10 degrees Celsius, 5-15 degrees Celsius). Cooling may be provided by the same instrument used to deliver thermal energy (e.g., heat) or a separate instrument. In accordance with several implementations, cooling is not used.

In some implementations, ablative cooling may be applied to the nerves or bone tissue instead of heat (e.g., for cryoneurolysis or cryoablation applications). The temperature and duration of the cooling may be sufficient to modulate intraosseous nerves (e.g., ablation, or localized freezing, due to excessive cooling). The cold temperatures may destroy the myelin coating or sheath surrounding the nerves. The cold temperatures may also advantageously reduce the sensation of pain. The cooling may be delivered using a hollow needle under fluoroscopy or other imaging modality.

In some implementations, one or more fluids or agents may be delivered to a target treatment site to modulate a nerve. The agents may comprise bone morphogenetic proteins, for example. In some implementations, the fluids or agents may comprise chemicals for modulating nerves (e.g., chemoablative agents, alcohols, phenols, nerve-inhibiting agents, or nerve stimulating agents). The fluids or agents may be delivered using a hollow needle or injection device under fluoroscopy or other imaging modality.

One or more treatment devices (e.g., probes) may be used simultaneously or sequentially. For example, the distal end portions of two treatment devices may be inserted to different locations within a vertebral body or other bone or within different vertebral bodies or bones. Radiofrequency treatment probes may include multiple electrodes configured to act as monopolar, or unipolar, electrodes or as pairs of bipolar electrodes. The treatment device(s) may also be pre-curved or curveable such that the curved stylet is not needed or may have sharp distal tips such that additional sharpened stylets are not needed. In some implementations, any or all of the access tools and the treatment devices are MR-compatible so as to be visualized under MR imaging.

The one or more treatment devices (e.g., probes such as radiofrequency probes, treatment device 501 of a kit or system) may include an indicator configured to alert a clinician as to a current operation state of the treatment device. For example, the indicator may include a light ring disposed along a length of, and extending around a circumference of, the treatment device. The light ring may be configured to light up with different colors and/or exhibit other visible effects (e.g., pulsing on and off with certain patterns). The one or more treatment devices may also be configured to provide audible alerts (e.g., beeps having a certain frequency or intonation) corresponding to different operational states. In one implementation, the light ring may be dark or not lit up when the treatment device is not connected to a radiofrequency generator or not ready for RF energy delivery. The light ring may pulse at a first rate (e.g., 1 pulse every 2-3 seconds) to indicate an operational state in which the treatment device and generator system are ready to initiate RF energy delivery. The light ring may be continuously lit up to indicate an operational state in which the treatment device is actively delivering RF energy. The light ring may pulse at a second rate different than (e.g., faster than, slower than) the first rate to indicate an operational state in which an error has been detected by the generator or if a particular treatment parameter is determined to be outside an acceptable range of values. In one implementation, the second rate is greater than the first rate (e.g., 2 pulses per second). Haptic feedback may also be provided to the clinician for at least some of the operational states to provide a further alert in addition to a visible alert.

In some implementations, the treatment device (e.g., treatment device 501) includes a microchip that is pre-programmed with treatment parameters (e.g., duration of treatment, target temperature, temperature ramp rate). Upon electrical connection of the treatment device to the generator, the treatment parameters are transmitted to the generator and displayed on a display of the generator to provide confirmation of desired treatment to a clinician.

Access to Locations Outside Vertebral Body

For access to locations outside bone (e.g., extraosseous locations, such as outside a vertebral body), visualization or imaging modalities and techniques may be used to facilitate targeting. For example, a foramen of a vertebral body (e.g., basivertebral foramen) may be located using MRI guidance provided by an external MR imaging system, CT guidance provided by an external tomography imaging system, fluoroscopic guidance using an external X-ray imaging system, and/or an endoscope inserted laparoscopically. Once the foramen is located, therapy (e.g., heat or energy delivery, chemoablative agent delivery, cryotherapy, brachytherapy, and/or mechanical severing) may be applied to the foramen sufficient to modulate (e.g., ablate, denervate, stimulate) any nerves entering through the foramen. For example, an endoscope may be used to locate the foramen under direct visualization and then the basivertebral nerve may be mechanically transected near the foramen. In some implementations, an intervertebral disc and vertebral body may be denervated by treating (e.g., ablating) a sinuvertebral nerve prior to the sinuvertebral nerve branching into the basivertebral nerve that enters the basivertebral foramen of the vertebral body.

Robotically-Assisted Access and/or Treatment

Access to and/or treatment within or adjacent bones (e.g., vertebral bodies) may be facilitated by the use of robotic navigation systems or robotically-controlled devices (e.g., computer-aided or computer-assisted systems or devices). For example, robotics may be used to facilitate or assist in positioning, targeting, deployment (e.g., hammering or malleting) so as to avoid over-insertion that might cause injury or damage, and/or to facilitate nerve sensing. FIG. 4 schematically illustrates an example of a robotically-enabled system 700. The robotic system 700 may be a robotic control, surgical, and/or navigation system capable of performing a variety of medical and/or diagnostic procedures and/or providing guidance and enhanced imaging to a clinician. The robotic system 700 may be a robotic assisted spinal surgery system, or a spinal robotics system.

The robotic system 700 may include an operator workstation or control console 702 from which a clinician can control movement of one or more robotic arms 703 to provide improved ease of use and fine control of movement. The workstation or control console 702 may include a computer-based control system that stores and is configured to execute (e.g., using one or more processors) program instructions stored on a non-transitory computer-readable storage medium (e.g., solid state storage drive, magnetic storage drive, other memory).

The robotic arms 703 may be configured to move with six or more degrees of freedom and to support or carry the access tools, treatment devices, and/or diagnostic devices. The robotic arms 703 may include multiple separate arm portions connected to each other at joints or pivot points so as to facilitate movement with six or more degrees of freedom along various axes of rotation. The robotic arms 703 may comprise multiple separate arms each separately controllable and each configured to hold and maneuver a different surgical instrument (e.g., access tool or treatment device). The robotic arms 703 may be coupled to a support system and controlled by one or more instrument drive systems that are in turn controlled by the control console 702. The instrument drive systems may include electro-mechanical components and mechanisms (e.g., drive motors, gears, pulleys, joints, hydraulics, wires, etc.) configured to actuate and move the robotic arms 703.

The robotic system 700 may also include one or more imaging devices 704 (cameras, endoscopes, laparoscopes, ultrasound imaging modality, fluoroscopic imaging modality, CT imaging modality, MR imaging modality, and/or the like). The imaging devices 704 may be supported or carried by one or more of the robotic arms 703. The imaging devices 704 may be components of an imaging system that facilitates 360-degree scanning of a patient. The imaging devices 704 may include stereotactic cameras, optical sensors, and/or electromagnetic field sensors. In some implementations, the imaging devices 704 of the robotic system 700 reduce an amount of patient exposure to radiation. The imaging devices 704 may be calibrated to patient anatomy or using reference pins or trackers positioned at one or more locations of the patient's body by a registration, or localization, system. The registration system may include multiple computing devices (e.g., processors and computer-readable memory for storing instructions to be executed by the processor(s)). The registration may involve identification of natural landmarks of one or more vertebrae (e.g., using a pointer device of the registration system).

The imaging system may be configured to communicate with software (e.g., running on the operator workstation or control console 702 or the registration system) that is configured to generate a real-time 3D map that may be registered with the robotic arms 703 or instruments carried by the robotic arms 703. The software may include surgery planning software configured to plan, based on 2D, 3D, or 4D pre-operative images (e.g., obtained via CT, MRI, fluoroscopy, or other imaging modalities) a desired trajectory for access to a target treatment location within a vertebral body or other bone. However, pre-operative planning may not be used in some implementations and navigation may be performed intraoperatively. The software may include navigation software configured to control the robotic arms 703 and provide feedback regarding navigation (e.g., trajectory and positioning information) to an operator at the operator workstation or on a separate display device (e.g., a display on an augmented reality headset or eyewear or tablet). A computing device of the control console 702 is configured to direct movement of the robotic arms 703 based on instructions executed by the computing device (either via inputs (e.g., manual joystick controls or voice-activated commands) from a clinician or via automated programs and artificial intelligence algorithms stored in memory). The computing device includes one or more specialized processors. The robotic system 700 may be used to carry out any of the methods of access, diagnosis, or treatment described herein while providing controlled movements to reduce likelihood of injury caused by manual operator error or error in judgment.

In some implementations, the robotic system 700 includes a closed-loop system that alters trajectory of access tools or treatment devices based on feedback. The feedback may include feedback based on the use of artificial intelligence or deep learning models based on neural networks that have been trained using supervised or unsupervised training techniques involving a large database of pre-operative and post-operative patient images having received a particular neuromodulation treatment. The images may involve patients having vertebral bodies of different bone structures (e.g., bone densities), bone anatomies, and degradation or defects (e.g., pre-Modic change characteristics such as vertebral endplate degeneration or defects or diagnosed Modic changes). The feedback may be based only on images of the particular level of vertebral body being treated, images of vertebral bodies having similar density as the vertebral body being treated, images of vertebral bodies from patients having similar patient characteristics (e.g., age, lifestyle, gender, pain scores). The neuromodulation (e.g., optimum neuromodulation parameters) may also be robotically implemented based on intelligent (e.g., artificial intelligence) feedback. The robotic system 700 may include a machine-driven navigation system deploying an energy source towards a target within a vertebral body to be treated. Detection and monitoring of the energy source's proximity to the target may be provided by the one or more imaging devices. The robotic system 700 can independently modify the trajectory and/or neuromodulation parameters) in response to imaging or other registration modalities. Modification of the trajectory may be via change in the configuration of a driving system (e.g., robotic arms 703) and/or by change of the configuration of the energy delivery device or assembly. Modification of trajectory may be automatic (e.g., closed-loop) or based on a feedback mechanism to an operator (e.g., open-loop). The open-loop mode may include boundary conditions (e.g., haptic conditions) or not. The detection and monitoring functions may rely on pre-operative and/or intra-operative data. Registration and targeting may be a priori or interactive.

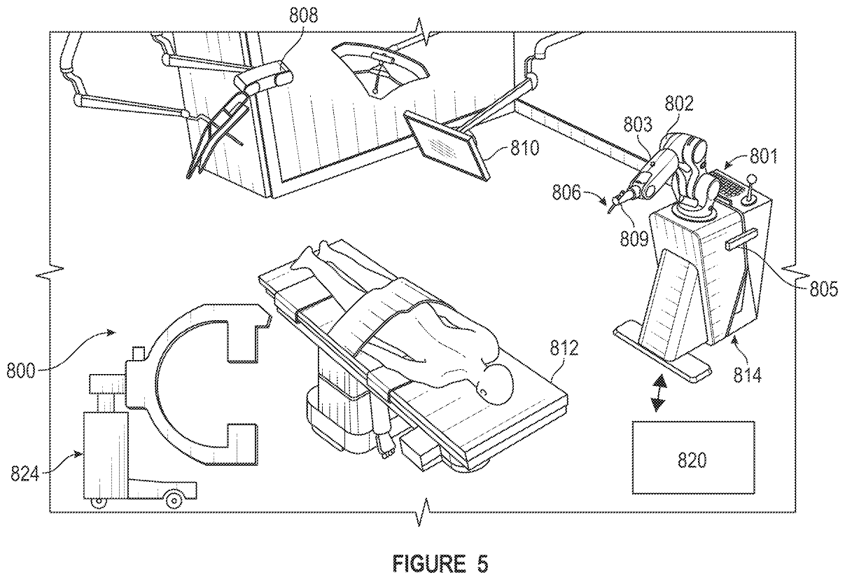

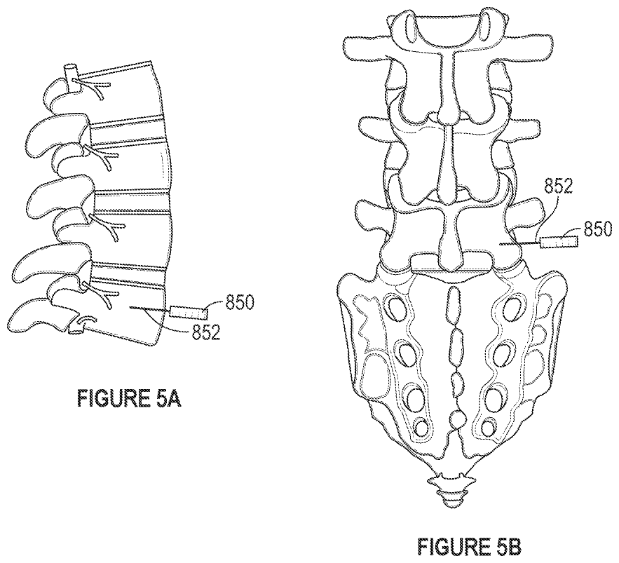

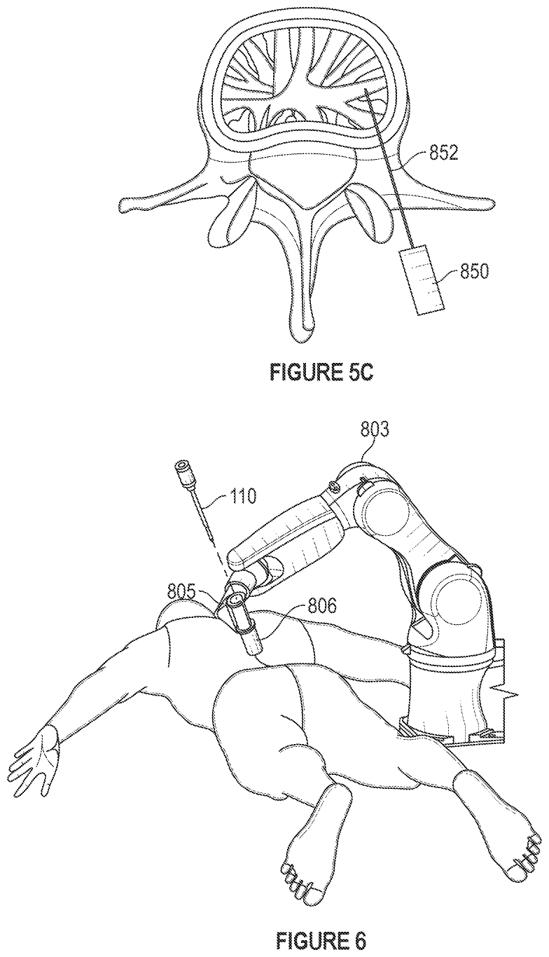

FIG. 5 illustrates another example robotically-enabled system 800 that can be used to facilitate or assist in positioning, targeting and/or deployment (e.g., hammering) of one or more of the access or surgical tools described above. In certain applications, the robotically-enabled system 800 can provide a guide to the surgeon to aid in accurately executing one or more of the methods for modulating nerves within or adjacent (e.g., surrounding) bone such as, an intraosseous nerve (e.g., basivertebral nerve or other nerve(s) within a vertebral body or nerve(s) that innerve a vertebral endplate) within a bone. For example, the robotically-enabled system 800 can provide robotic guidance of the surgical tools (e.g., one or more of the surgical tools, bone access tools, or treatment devices described herein) to ensure proper trajectory of the tool (e.g., the introducer assembly 110, the curved cannula assembly 210, the introducer drill 440, and/or the treatment device 501).

With reference to FIG. 5, the robotic system 800 may include an operator workstation or control console 801 from which a clinician can control movement of a tool holder to provide improved ease of use and fine control of movement. The workstation or control console 801 can include a computer-based control system that stores and is configured to execute (e.g., using one or more processors) program instructions stored on a non-transitory computer-readable storage medium (e.g., solid state storage drive, magnetic storage drive, other memory). The control console 801 can include various instruments for a user to provide input to the control system such as a joystick or other handheld controller and/or keyboard as illustrated in FIG. 5.

In certain implementations, the surgical robotic system 800 includes a surgical robot 802 on a mobile cart 814. The surgical robot 802 can be positioned near an operating table 1812 without being attached to the operating table 812. In the illustrated arrangement, the control console 801 is also on the mobile cart 814. In certain implementations, the control console 801 and the mobile cart 814 can be separate units and/or the surgical robot 802 can be stationary. For example, in certain configurations, the surgical robot 802 is securable to the operating table 812 or formed as part of the operating table 812. For example, in certain implementations, the surgical robot 802 can be positioned on a rail system (not illustrated) positioned on the operating table and/or positioned above the operating table 812.

The mobile cart 814 may permit a user (operator) to move the surgical robot 802 to different locations before, during, and/or after a surgical procedure and/or allow the surgical robot 802 to be transported into and out of the operating room 803. The mobile cart 814 may include wheels and/or be positioned on a track system. The mobile cart 814 can also include a handle 805 such that the mobile cart 814 can be moved by a user. In certain implementations, the cart 814 can be motorized and the position of the cart 814 can be controlled remotely by a user.

The cart 814 can include a stabilization system that can be used to increase the stiffness of the cart 814 to aid in the accuracy of the surgical procedure. For example, in certain implementations, the wheels can include a locking or braking mechanism that prevents the cart 814 from moving after being placed in a locked or braked configuration. In certain arrangements, the stabilizing, braking, and/or locking mechanism can be activated when the surgical robot 802 is turned on. The stabilizing, braking, and/or locking mechanism(s) can be entirely mechanically and/or electronically activated and deactivated and/or can be manually and/or automatically activated and deactivated.

As shown in FIG. 5, the surgical robot 802 can include a robotic arm 803 mounted on the mobile cart 814. The robotic arm 803 can be configured to move with one or more degrees of freedom (e.g., four degrees of freedom with two degrees of translation and two degrees or rotation or six degrees of freedom with three translations and three rotations) and to support or carry an end effector or tool holder 809 that holds a surgical tool 806 which can be a surgical guide 806 as described below. The arm 803 may include multiple arm portions connected at pivot joints to facilitate movement about the multiple degrees of freedom or rotation. The arm 803 can include electro-mechanical components and mechanisms (e.g., motors gears, pulleys, joints, hydraulics, wires, etc.) configured to actuate and move the robotic arm 803.

In certain configurations, the end effector 809 of the robotic arm 803 is configured to releasably hold the surgical tool 806 (e.g., access tool, treatment device, diagnostic device, a surgical guide), allowing the surgical tool 806 to be removed and replaced with a second surgical tool. The system 800 may allow the surgical tools (e.g., access tools or treatment devices described herein) to be swapped without re-registration, or with automatic or semi-automatic re-registration of the position of the end-effector 809.

As shown in the illustrated example of FIG. 5, the system 800 can include a tracking system 808 that can be used to capture the position of the patient and different components of the surgical robot 802, and a display screen 810 that displays, for example, real time patient data and/or real time surgical robot trajectories. The tracking system 808 can be used to monitor the location of patient and the surgical robot 802. The tracking system 808 can include one or more cameras, video cameras, infrared detectors, localizers, field generators and/or sensors for electro-magnetically and/or optically tracking the robot 802 and/or the patient. The display screen 810 can also be coupled to the cart 814 and/or multiple display screens can be provided. In certain implementations, the display screen 810 may be incorporated into or duplicated on one or more headsets worn by users. In certain implementations, the headsets with display screens (e.g., heads-up displays) can be augmented reality headsets or eyewear or virtual reality headsets and the system 800 is configured for the operators to operate in an augmented reality or virtual reality environment. The display screen 810 may also include a see-through display on a tablet or portal that is configured to be positioned just above a surgical access location.

In certain arrangements, the robotic system 800 can be used with an augmented reality device 820 (such as but not limited to Google Glass, Microsoft's HoloLens headsets, or Osterhout Design Group's augmented reality smart glasses and virtual reality headsets) to display an optimal position of the surgical instruments positioned by the robotic system 800. In certain configurations, the glasses or headsets can include an optical head-mounted display. An optical head-mounted display (OHMD) can be a wearable display that has the capability of reflecting projected images as well as allowing the user to see through it. The augmented reality device 820 may alternatively comprise a tablet or portal that can be coupled to an arm of the robotic system 800 or coupled to a patient operating table or stand so that the tablet or portal can be placed just above the target treatment site.

A silhouette of the tool (e.g., surgical tool such as an access tool or treatment device) and/or the target site at the optimal position may be shown in the field of view of the augmented reality device 820. A positioning indicator may also be shown in the field of view to guide or instruct positioning. The augmented reality device 820 may collect information either from the internal sensors (e.g., optical sensors, electromagnetic sensors, ultrasound sensors, force sensors, motion sensors, proximity sensors) within the robotic system 800 and/or the tracking system 808. In certain configurations, the robotic system 800 is configured to operate in both a tracking and a non-tracking mode and can include a head mounted display configured to provide an optical view of a patient and to inject or project received data content over top of the optical view to form an augmented reality view of the patient, and comprising internal tracking devices configured to determine the position of surgical instruments positioned by the robotic system 800 relative to the patient and the target anatomy and/or treatment site.

The augmented reality device 820 may include a see-through display configured to display virtual images overlaid or superimposed on top of a real-time field of view of the surgical tools and patient such that the orthopedic surgeon does not have to turn his or her head to view one or more displays or written procedural instructions outside of the procedural field of view. The virtual images may provide surgical or procedural guidance overlaid or superimposed on the optical field of view. The virtual images may include real-time video or imaging feed (e.g., fluoroscopic or computed tomography image guidance) of the target treatment site (such as a vertebral body); a planned virtual trajectory or path displayed as an indicator, icon, line, arrow, or other image; pre-operative 2D, 3D or 4D anatomical images (e.g., magnetic resonance or computed tomography images); and/or alphanumeric content (e.g., procedural instructions, cautions, warnings, alerts, real-time or previously-obtained characteristics (such as heart rate, bone density measurements, vertebral body level indicators or labels (e.g., L4, L5, S1), and/or real-time treatment parameters (e.g., impedance measurements, treatment duration timer, temperature measurements, power output measurements)). In some implementations, intraoperative 3D imaging technology is not used.

The operator may also input text or annotations on the display of the augmented reality device 820 using voice-activated commands or via a virtual touch-screen keyboard on the display or via one or more input devices (e.g., joysticks) wirelessly coupled to the augmented reality device 820. The inputted text or annotations may be reproduced on other displays within a hospital operating room, ambulatory surgical center, or outpatient procedure room for others to view. The inputted text or annotations may be recorded and stored with the intra-operative video or still images (e.g., for documentation or follow-up purposes). The augmented reality device 820 may also allow operators to perform procedures from remote locations over a communications network. The augmented reality device may be communicatively coupled to the robotic system 800 so that the operator can control operation of the robotic system 800 from an in-person location or from a remote location.

The robotic system 800 can further include an augmented reality computing system (not shown) comprising one or more processors, one or more computer-readable tangible storage devices, and program instructions stored on at least one of the one or more storage devices for execution by at least one of the one or more processors. In certain configurations, a stereoscopic augmented view of a patient from a static or dynamic viewpoint of the surgeon or other clinician, which employs real-time three-dimensional surface reconstruction for preoperative and intraoperative image registration can be provided. In such configurations, stereoscopic cameras can provide real-time images of the scene including the patient. A stereoscopic video display or display on the augmented reality device 820 can be used by the surgeon to see a graphical representation of the preoperative or intraoperative images blended with the video images in a stereoscopic manner through a see-through display. While embodiments of the controllers and displays above are discussed in the context of augmented reality applications, teachings disclosed herein can be used in connection with other digital reality applications, such as virtual reality, mixed reality, etc. As such, the discussion of augmented reality is for illustrative purposes only and does not limit this disclosure to augmented reality applications.

For example, the robotic system 800 may be integrated with a virtual reality system to facilitate training of surgeons or other clinicians on performing neuromodulation procedures (e.g., intraosseous nerve ablation procedures described herein) using the access tools and treatment devices described herein. A virtual reality system for providing virtual reality environment may include a processor (e.g., a processor configured to implement computer-executable instructions stored in memory) for generating a virtual reality environment (e.g., a minimally-invasive spinal neuromodulation, such as basivertebral nerve ablation, virtual reality environment). The virtual reality system may also include a head-mounted display (e.g., within a headset or googles) wearable by an operator, and one or more handheld controllers (e.g., joysticks) that can be manipulated by the operator for interacting with the virtual environment provided by the virtual reality system.

In some implementations. the head-mounted display includes an immersive virtual reality display for displaying the virtual environment to the operator (e.g., with a first-person perspective view of the virtual environment). The virtual reality system can additionally or alternatively include one or more external displays for displaying the virtual environment to others in addition to the operator (e.g., for training or evaluation purposes). The immersive display and the one or more external displays may be duplicative of one another, or synchronized, so as to show the same content. In accordance with several configurations, the virtual reality system may be configured to generate a virtual environment within which an operator (e.g., surgeon or other clinician) may navigate around a virtual operating room (e.g., within an outpatient operating room or ambulatory surgical center) and interact with virtual patients and surgical tools (e.g., bone access instruments, diagnostic tools, treatment devices that are typically used in a spinal neuromodulation procedure) via the head-mounted display and/or handheld controllers.

The virtual reality system may be used to prepare for performing spinal neuromodulation procedures (e.g., basivertebral nerve ablation procedures) using robotic systems, including but not limited to training, simulation, and/or collaboration among multiple surgeons, students, researchers, or other clinicians.

In some implementations, a virtual reality system may interface with an actual real-life operating room. In some implementations the virtual reality system provides visualization (via the head-mounted display) of a robotic spinal neuromodulation environment, and may include one or more computer processors configured to generate a virtual robotic spinal neuromodulation environment that includes at least one virtual robotic arm or robotic manipulator, and at least one sensor or tracker (e.g., optical sensors, electromagnetic sensors, ultrasound sensors, force sensors, motion sensors, proximity sensors). The sensor or tracker may be communicatively coupled with the one or more computer processors and may be configured to detect a status of an actual robotic arm or robotic manipulator corresponding to the virtual robotic arm or robotic manipulator. The one or more processors may be configured to receive the detected status of the robotic arm or robotic manipulator and modify the virtual robotic arm or robotic manipulator based on the detected status such that the virtual robotic arm or robotic manipulator copies or mimics the actual robotic arm or robotic manipulator.

As one example, a trainer or observer (e.g., student or peer clinician) may monitor an actual robotic spinal neuromodulation procedure in an actual operating room via a virtual reality system that interfaces with the actual operating room (e.g., the trainer or observer may interact with a virtual reality environment that reflects the conditions in the actual operating room).

With continued reference to FIG. 5, the robotic system 800 may also include one or more imaging devices 824 (cameras, endoscopes, laparoscopes, ultrasound imaging modality, fluoroscopic imaging modality, MR imaging modality, and/or the like). The imaging devices 804 may be supported or carried by the robot 802. The imaging devices 824 may be components of an imaging system that facilitates 360-degree scanning of a patient. The imaging devices 824 may include stereotactic cameras and/or electromagnetic field sensors. The imaging devices 824 may be calibrated to patient anatomy or using reference pins or trackers positioned at one or more locations of the patient's body by a registration, or localization, system. The registration system may include multiple computing devices (e.g., processors and computer-readable memory for storing instructions to be executed by the processor(s)). The registration may involve identification of natural landmarks (e.g., spinous process, superior articular process, transverse process, pedicle, basivertebral foramen) of one or more vertebrae (e.g., using a pointer device of the registration system). Trackers and/or tactile sensors may be attached to external skin locations corresponding to locations of one or more spinous processes of the vertebrae to help determine shapes and/or locations of the one or more spinous processes, which shapes or locations can then be matched to a three-dimensional model of the spine (or a portion of the spine) for registration purposes.

In certain implementations, the display screen 810 can be used to display a projected or virtual trajectory and/or a proposed trajectory of a tool being inserted through the tool holder 806 wherein the virtual trajectory forms a virtual axis. By continuously monitoring the patient and robotic arm positions, using tracking detector 808 and/or internal sensors (e.g., optical encoders and/or other position sensors) positioned within the surgical robot 802, the surgical system can calculate updated trajectories and visually display these trajectories on the display screen 810 to inform and guide operators (e.g., surgeons and/or technicians) in the operating room using the surgical robot. In addition, in certain implementations, the surgical robot 802 may also change its position and automatically position itself based on trajectories calculated from the real time patient and robotic arm positions captured using the tracking detector 808. For instance, the trajectory of the end-effector can be automatically adjusted in real time to account for movement of the vertebrae and/or other part of the patient 104 and/or movement of the arm 803 during the surgical procedure.

As noted above, the imaging devices 824 may be calibrated to patient anatomy or using reference pins or trackers positioned at one or more locations of the patient's body by a registration, or localization, system. The registration system may include multiple computing devices (e.g., processors and computer-readable memory for storing instructions to be executed by the processor(s)). The registration may involve identification of natural landmarks (e.g., spinous process, superior articular process, transverse process, pedicle, basivertebral foramen) of one or more vertebrae (e.g., using a pointer device or the registration system). The imaging system may be configured to communicate with software (e.g., running on the operator workstation or control console or the registration system) that is configured to generate a real-time 3D map that may be registered with the robotic arms 803 or instruments carried by the robotic arms 803. The software may include surgery planning software configured to plan, based on pre-operative images (e.g., obtained via CT, MRI, fluoroscopy, or other imaging modalities) a desired trajectory for access to a target treatment location within a vertebral body or other bone. The desired trajectory or path may be suggested by the surgery planning software. However, pre-operative planning may not be used in some implementations and navigation may be performed intraoperatively.

The desired trajectory may be generated automatically via automated surgical planning, may be generated using machine-learning algorithms involving trained neural networks, or may be determined by a surgeon or other clinical professional. The desired trajectory may be based on one or more factors or parameters, such as a bone density of a target vertebra, whether access will be performed transpedicularly or extrapedicularly, the level of the vertebra (e.g., sacral, lumbar, thoracic, cervical, S1, S2, S3, S4, L1, L2, L3, L4, L5, etc.), the location of the basivertebral foramen, a preset curvature of the access instrument, whether access will be performed using straight instruments or pre-curved instruments or steerable instruments, and/or other factors or parameters. The desired trajectory may also be based on preoperative or intraoperative patient images and/or past experience of the operator. The software may include navigation software configured to control the robotic arms 803 and provide feedback regarding navigation (e.g., trajectory and positioning information) to an operator at the operator workstation or on a separate display device. A computing device of the control console is configured to direct movement of the robotic arms 803 based on instructions executed by the computing device (either via inputs (e.g., joystick controls) from a clinician or via automated programs and artificial intelligence algorithms stored in memory). The computing device includes one or more specialized processors. The robotic system 800 may be used to carry out any of the methods of access, diagnosis, or treatment described herein while providing controlled movements to reduce likelihood of injury caused by manual operator error or error in judgment. In some implementations, the robotic system 800 includes a closed-loop system that alters trajectory of access tools or treatment devices based on feedback (e.g., artificial intelligence using one or more trained neural networks or other machine learning methods).

FIGS. 5A and 5B illustrate lateral and posterior views respectively of lower segments of the spine. In certain implementations, these views which may be live and/or recently captured images (e.g., fluoroscopic views) of the spine captured by the imaging system 824 may be displayed on the display screen 810 to aid in guidance. In certain configurations, the system 800 is configured to display actual images or graphical representations 850 of the surgical tool 806 projected onto the lateral and posterior views. In addition, the system 800 can display trajectory aids such as projection lines 852. In this manner, the display screen 810 can be used to display a projected trajectory and/or a proposed trajectory of a tool being inserted through the tool holder 806.

FIG. 5C illustrates an image which can be created by the system 800 to aid in navigation. The image of FIG. 5C can correspond to cross-sectional top image through a vertebra. The system 800 can be configured to create and display this view from the captured images from the imaging system 800 and/or models of the spine (e.g., preoperative models). In addition, the system 800 can display trajectory aids such as projection lines 852. In this manner, the display screen 810 can be used to display a projected trajectory and/or a proposed trajectory of a tool being inserted through the tool holder 806.

The system 800 can allow a surgeon to physically manipulate the tool holder 806 to safely achieve proper alignment of a tool insert through the tool holder 806 for performing crucial steps of the surgical procedure (e.g., initial insertion steps that affect the eventual trajectory of subsequent access tools, which may be pre-curved). Operation of the robotic arm 803 by the surgeon (or other operator) in force control mode may permit movement of the tool in a measured, even manner that disregards accidental, minor movements of the surgeon. In this manner, the surgeon can move the tool holder 806 to achieve proper trajectory of the tool (e.g., a stylet) prior to operation or insertion of the tool into the patient. Once the robotic arm 803 is in the desired position, the arm 803 and the tool holder 806 in turn can be fixed to maintain the desired trajectory. The tool holder 806 can then serve as a stable, secure guide through which a tool may be moved through or slid at an accurate angle.

FIG. 6 illustrates an example configuration of the robotic arm 803. The robotic arm 803 can include an end-effector 804, which in turn can hold a surgical instrument guide 806. The manipulator (not shown in FIG. 6), such as a handle on the robotic arm 803 and/or near the end effector and/or remote from the cart, can be used by a surgeon or other clinician for robotically-assisted and/or unassisted positioning and/or movement of the surgical instrument guide 806 by a user to align an axis defined by the instrument guide 806 at a desired trajectory in relation to a patient situation. The axis can be aligned with the desired trajectory in relation to the patient situation via the manipulator using the display (e.g., display of an augmented reality device 820) to aid in obtaining the desired trajectory as described above.

The surgical instrument guide 806, is coupled to the robotic arm 803 for guiding instruments during surgery. For example, the surgical instrument guide 806 may be coupled to the robotic arm 803 via a flange. The surgical instrument guide 806 is configured to hold and/or restrict movement of a surgical instrument (e.g., an introducer assembly, stylet drill, or treatment device) therethrough. As shown in FIG. 6, in some implementations, the surgical instrument guide 806 comprises a cannula through which tools such as the introducer assembly 110, curved cannula assembly 210, and treatment device 501 described above can be inserted.

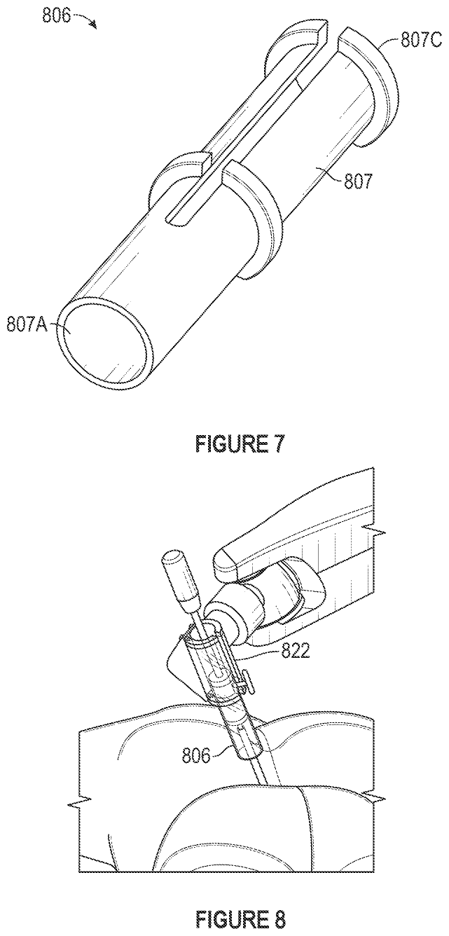

FIG. 7 illustrates in more detail an example configuration of the surgical instrument guide 806 for use with a robotic surgical system 800. In some implementations, the same surgical instrument guide 806 is used to guide all the instruments utilized with a robotic surgical system. For example, once the desired trajectory is selected, the robotic system 800 may not move during the steps of accessing bone (e.g., the vertebral body) and then modulating nerves (e.g., basivertebral nerve and/or other intraosseous nerves) within or adjacent (e.g., surrounding) the bone (e.g., vertebral body). Such steps may include inserting access tools such as an introducer assembly including an outer cannula and a sharpened stylet, an inner cannula configured to be introduced through the outer cannula, and/or one or more additional stylets, curettes, or drills to facilitate access to an intraosseous location within a vertebral body or other bone. A treatment device 501 can then be inserted through the introducer assembly to modulate (e.g., ablate or stimulate) nerves. The paths may be curved or straight.

With continued reference to FIG. 7, the surgical instrument guide 806 can include a rigid hollow tubular structure 807 having a first open end 807A and a second open end 807B. The surgical guide 806 can include one or more flanges that can be configured to secure the guide 806 to an end effector of the robotic surgical system. The tubular structure 807 can define an insertion axis along surgical instrument sliding through the structure 807 is restricted. That is, in certain configurations, the tubular structure 807 can constrain or limit movement except along the longitudinal axis defined by the tubular structure 807. This insertion axis can correspond to the projected trajectory and/or a proposed trajectory shown on the display (e.g., display of an augmented reality device) as described above.

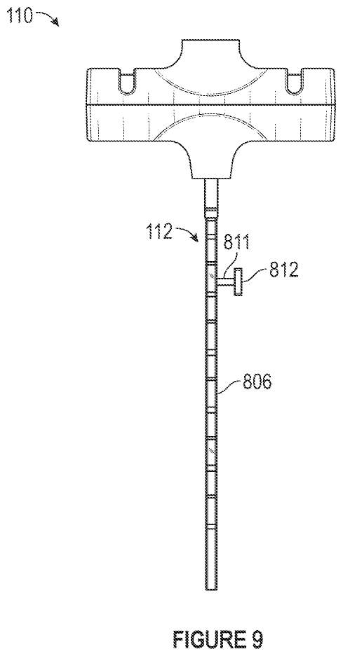

FIG. 8 illustrates a surgical instrument sliding through the surgical instrument guide 806. As discussed above, the guide 806 can include a notch 822 that extends along the length of the guide 806. The surgical instrument (e.g., any one of the surgical instruments described herein, such as access tools or treatment devices) can include an alignment pin that can slide through the notch 822. In this manner, the notch 822 permits the surgical instrument to slide along the axis defined by the guide while the guide is held in a fixed position by the robotic surgical system 800. In certain implementations, a navigation marker 812 can be provided on the alignment pin such that the navigation maker 812 can be viewed by a navigation camera along an entire range of movement of the surgical instrument through the guide 806. FIG. 9 illustrates the introducer assembly 110 which can be modified to include an alignment pin 811 and marker 812. As described above, the alignment pin 811 can be used to slide the introducer assembly 110 along the axis defined by the guide 806 while the guide 806 is held in a fixed position by the robotic surgical system 800.

The guide 806 and the tool 811 can include various sensors and markers that are configured to provide an indication of the relative depth that the tool 811 has been inserted into the guide. This information can be used by the system 800 to aid in determining the position of the tool 811 with respect to the anatomy of the patient. In certain implementations, the tool may include a sensor or marker near or in proximity to an end of the tool and the position of the sensor or marker can be measured by the system and displayed on the display (e.g., display of an augmented reality device) to aid the operator.

In certain applications, the robotic system 800 can use automatic planning (e.g., closed-loop mechanism). For example, in certain applications, the system 800 can be used to obtain patient medical images. These medical images can come from MRI, CT, fluoroscopy, CT, or 3D fluoroscopy (or a combination of images from multiple image modalities). These images can be obtained pre-operatively and fed into the system 800, as it is often the case for MRI or CT, or intra-operatively which is often the case for fluoroscopy, ultrasound and 3D fluoroscopy. The patient can then be registered by finding and correlating the actual patient anatomy with the medical images. This can be done automatically for intra-operative medical imaging. For example, a patient navigation marker can be attached to the vertebrae or other portion of the spine or patient before the pre-operative images are taken. The patient navigation marker can be recognized on the images by the software and by knowing the position of the imaging device, medical images can be related to the position of the patient navigation marker for further use. Other approaches for registering the patient can include manual point-to-point registration, surface matching, and fluoroscopy-based registration (e.g., intraoperative fluoroscopic images are matched to pre-operative CT images).

For certain systems, the trajectory can be planned automatically based on medical images obtained pre-operatively or intra-operatively (from one or more imaging modalities). An automatic planning program can be configured to take pre-operative or intra-operative medical images as an input and based on these medical images recognize the vertebrae and proposes the most suitable trajectories and or propose an initial trajectory which can then be modified by the user. The automatic planning program may recognize sensitive anatomy (e.g., spinal cord tissue or nerves not intended to be treated) and display warning indicators if the proposed trajectory would encounter such sensitive anatomy and/or violated certain surgical guidelines.

Modified methods of selecting the trajectory can be based on user specifying a target or end point within a patient's vertebrae and the system automatically presenting proposed trajectory/trajectories based on certain rules or guides or based on known pre-set curvatures of access tools and/or known or measured densities of bone. In another example, the user can position the robotic arm 803 whose position is known to the system 800 and the tool axis through the surgical guide tool at the end of the arm can be projected on to the images as described above with respect to FIGS. 5A and 5B. As noted above, the surgeon can be provided with a virtual projection of the trajectory thereon (e.g., on a display of the robotic surgical system or an augmented reality device). Typically, a surgeon uses lateral and anterior-posterior medical views to review the trajectory (but other and/or additional views can be used as well).

Once a trajectory is chosen, in certain implementations, the robot can automatically move the surgical guide tool to the trajectory following user instruction. In other implementations, the user uses the display and manually controls movement of the robot such the surgical guide or tools inserted therein are positioned along the selected.

In certain arrangements, the robot system 800 is equipped with a force sensor. The force sensor can be used to identify collisions by an increase of forces (e.g., if the force sensor is expecting to measure no force, the system can detect a collision if a force is detected). Similarly, additional sensors (e.g., artificial skin, laser scanner, electrical/capacitive proximity sensors, etc.) can be added on the robotic arm 803 with a goal of detecting a collision. Upon detecting a collision, the robot may react accordingly, such as stopping or adapting movement.

As noted above, in some implementations, rather than using automatic movement, the surgeon/user moves the robot “manually”, as in hands-on planning (e.g., an open-loop mechanism). However, the robot may still provide the surgeon with aids in finding the trajectory by providing haptic feedback (e.g., force and/or torque). This assistance can be provided by simulating attractive forces/torques to guide the surgeon to bring the robot to the target position. In certain implementations, these forces/torques make it easier to move to the direction of correct trajectory while preventing/making difficult to move in the other direction (e.g., the latter, by providing resistive forces). For example, spring-like forces (proportional to the distance) or magnetic-like forces (proportional to square of the distance) may be used to guide the user to the trajectory. The haptic feedback may be delivered to the user (e.g., surgeon) by an actuator associated with the robotic arm, controlled by a processor. The amount (intensity) of force feedback to be delivered to the user may be computed in real-time as a function of the position of the robotic arm in relation to the computed correct trajectory. The haptic feedback may be computed and delivered in relation to a haptic guide, where the haptic guide provides constraints to particular regions, points, and/or surfaces, or the haptic guide may provide detents or force fields to encourage movement toward a particular position in 3D space at a particular orientation (yaw, pitch, roll).

As noted above, once the robot is along the correct trajectory, it can be locked into position. In aspects where the robot holds a surgical guide instrument, manually controlled instruments may be inserted through the surgical guide instrument. In other implementations, in which the surgical instruments are also controlled by robotic arms, the user is allowed to move the surgical instruments only along the trajectory (e.g., if it is a line in space) or rotate along the trajectory if the rotation of the tool is not important. In certain implementations, when a user tries to move out of the trajectory, a repulsive force is provided, thereby preventing the surgeon from moving the instrument out of trajectory. In a similar manner, motion along the length of the trajectory may be limited to prevent tools from being inserted too far into the patient. Various other feedback mechanisms can be used such as visual feedback (lights, changing colors, etc.), and/or an audio alarm when the surgeon moves out of the trajectory or advances a tool too far. For example, an alarm may display or sound if there is a danger of overdrive.

In certain implementations, once positioned along the trajectory, the tool held by the robotic arm 803 might become out of alignment with the trajectory, for example, due to movement of the patient (e.g., breathing), forces applied to the vertebrae, or movement of the whole table. In this case, the appropriate mode may be activated to provide assistance in finding the trajectory again to move the robot into the correct, new trajectory. In certain cases, the movement (e.g., of the patient or table) is measured and the robot reacts to the movement automatically (e.g., the system tracks the vertebra(e)). For example, the robotic system 800, in certain implementations, provides real-time compensation to follow the movement of the vertebra(e). Accordingly, in certain implementations, the robotic system 800 is configured to automatically adjust the position of the instruments based on change in the position of the vertebral body such that a spatial relationship between the surgical instruments and the vertebral body remains substantially unaltered as at least a portion of an operation of ablating a basivertebral nerve, or other neuromodulation operation, is performed. In certain configurations, the robotic system 800 automatically adjusts the position of the surgically instrument guide based on change in the position of the vertebral body such that a spatial relationship between the surgical instrument guide and the vertebral body remains substantially unaltered as at least a portion of an operation of ablating a basivertebral nerve or other intraosseous nerve within a vertebral body, or other neuromodulation operation, is performed.

In an example of a method for modulating nerves within or adjacent (e.g., surrounding) bone, and, in some implementations, an intraosseous nerve (e.g., basivertebral nerve) within a bone (e.g., vertebral body) of the spine is modulated for treatment, or prevention of, chronic back pain. The surgeon or other operator/assistant identifies a trajectory or planned path for a surgical tool (e.g., bone access introducer tool). In certain cases, the trajectory or path may be identified pre-operatively, intraoperatively, or a combination thereof. The trajectory may be based on pre-operative images of the particular patient's anatomy (e.g., one or more vertebral levels of the spine). The trajectory or path may be impacted by which vertebral body or vertebral bodies are being targeted (e.g., L4 vertebra, L5 vertebra, S1 vertebra). For example, the trajectory or planned path for a lumbar vertebra may be different than for a sacral vertebra. The trajectory or path may also be based on known or measured bone density or other bone structure characteristics of the patient. The trajectory or path may take into account the location of a desired treatment site within the vertebral body. For a transpedicular access approach, the trajectory or path may take into account that a curved approach may be required after exiting from the pedicle in order to access the location of the desired treatment site (e.g., predicted or known location of a basivertebral nerve trunk or other intraosseous nociceptive nerve that signals from the vertebral endplate). In some implementations, the trajectory is defined by a computer algorithm (e.g., based on a selected access approach, a level of the vertebral body, bone density of the vertebral body, bone structure of the vertebral body, particular patient anatomy surrounding the vertebral body, or other patient characteristics). The computer may define the trajectory or path with or without the surgeon's assistance. In some implementations, the trajectory or path is presented for the surgeon's approval. Once the planned or desired trajectory or path is set, the trajectory or path may be transferred to the robotic system 700, 800 for carrying out insertion of one or more access tools (e.g., introducer assembly and/or introducer drill) along the planned trajectory or path.

Next, the surgeon positions the end effector of a robotic arm (e.g., with the surgical guide) in accordance with the desired trajectory or path. The positioning may be assisted or unassisted by the robotic surgical system. In some implementations, the surgeon may be assisted using various types of indications such as visual and/or sound indications. The surgeon may be assisted by use of an augmented reality device display. The display of the augmented reality device may include a virtual image of the desired trajectory or path to facilitate proper alignment of the surgical tool with the desired trajectory or path. The display could also include a 2D, 3D or 4D virtual image of a portion of the patient's spine beneath the skin (e.g., a virtual 3D model based on pre-operative patient images) to provide an enhanced user experience with the surgeon “visualizing” (like “X-ray vision”) the portion of the patient's spine that requires treatment and to increase confidence in the trajectory or path. The visual or sound indications may be provided to the surgeon via the augmented reality device. The augmented reality device may be used with or without use of the robotic system but may require use in connection with a navigation, imaging, registration or localization system such as described above to determine the actual location of the surgical tool relative to the desired or planned trajectory or path.

After the position of the robotic arm 803 is fixed with the desired trajectory, a surgical instrument (e.g., the introducer assembly 110, curved cannula assembly 210, introducer drill 440 and/or treatment device 501) is maneuvered along the desired trajectory or path (e.g., in a manner that is constrained by the surgical instrument guide). For example, the distal portion of the introducer assembly 110 (including an alignment pin 811 and/or marker 814) can be inserted through a pedicle adjacent the vertebral body by advancing the introducer assembly 110 after insertion and aligned engagement of the introducer stylet 114 within the introducer cannula 112. The insertion may be monitored or supported by the surgeon using the display on the augmented reality device 820 to provide assurance of proper insertion. The display of the augmented reality device 820 may include a live video stream of a fluoroscopy imaging feed and/or still images or 3D models based on pre-operative anatomical images of the patient. The surgeon may control the advancement using controls (e.g., via joysticks or other handheld controllers, voice-activated commands) associated with the augmented reality device or with the robotic system 700, 800 that are communicatively coupled to the control system or control console 801 of the robotic system 700, 800). In some implementations, the robotic system 700, 800 may control advancement automatically without requiring operator control and the surgeon may just monitor the insertion by the robotic system 700, 800. The method may optionally include removing the introducer stylet 114 after initial penetration into the pedicle. The method may optionally include inserting the introducer drill 440 into and through the introducer cannula 112 (e.g., along the desired or planned trajectory or path) to complete the traversal of the pedicle and penetration through a cortical bone region of the vertebral body until a cancellous bone region of the vertebral body is reached. The insertion of the introducer drill 440 may be monitored or supported by the surgeon using the display on the augmented reality device 820 or automated similarly as described above for the introducer assembly 110. As noted below, one, some, or all of the tools inserted (e.g., through the surgical instrument guide) may include markers and/or sensors such that the robotic system 700, 800 can display (e.g., on a display of an augmented reality device) the position of the distal end of the tool to the user to aid the user in the surgical procedure. Because a robotic system 700, 800 is used, some of the access tools may not be required or needed due to the precision and control provided by the robotic system 700, 800. In addition, some of the access tools may be introduced using the robotic system 700, 800 and other access tools may be manually introduced by an operator.

After the cancellous bone is reached, the introducer drill 440 may be removed and the introducer stylet may be re-inserted within the introducer cannula 112 and advanced so as to advance the distal tip of the introducer cannula 112 to the entry site into (or within) the cancellous bone region of the vertebral body (e.g., along the desired or planned trajectory or path). The introducer stylet 114 may then be removed from the introducer cannula 112. In some implementations, fluoroscopic imaging is not used during the insertion of the introducer tools (e.g., introducer assembly 110 and/or introducer drill 440) by the robotic system 700, 800, thereby reducing radiation exposure.

The curved cannula assembly 210 may then be inserted within the introducer cannula 112 and advanced along the desired or planned trajectory or path. If the curved cannula assembly 210 is advanced by coupling it to the robotic system, the advancement may be adjusted either automatically by the robotic system if the actual path deviates from the planned trajectory or path. For example, the curved cannula assembly 210 may be partially or fully retracted and advanced again to more accurately follow the planned trajectory or path. In some implementations, the robotic system or the surgeon may decide that the current curved cannula assembly should be swapped out for a different curved cannula assembly having a different pre-set radius of curvature. In some implementations, a steerable or curveable channeling instrument may be used instead of the curved cannula assembly 210 so as to facilitate active adjustments to steering without relying on pre-curved devices.

A treatment device (e.g., a flexible bipolar radiofrequency probe) 501 may be inserted through the curved cannula 212 (e.g., after removal of the J-stylet 214 from the curved cannula 212) and advanced out of the open distal tip of the curved cannula 212 to the target treatment location. If a steerable channeling instrument was used instead of the curved cannula assembly, then the treatment device 501 may be inserted along the curved path formed by the steerable channeling instrument. The treatment device 501 may be inserted manually by the surgeon or other operator or automatically by coupling it to the robotic system 700, 800. The treatment device 501 may then be used to perform the desired treatment. For example, if the treatment device 501 is a radiofrequency probe, the treatment device 501 may be activated to ablate intraosseous nerves (e.g., a basivertebral nerve) or a tumor within the vertebral body. Bone cement or other agent, or a diagnostic device (such as a nerve stimulation device or an imaging device to confirm ablation of a nerve) may optionally be delivered through the curved cannula 212 after the treatment device is removed from the curved cannula.

A display of the augmented reality device 820 may be used by the surgeon or other operator to monitor the procedure (e.g., monitor treatment parameters from a display of a radiofrequency generator that may be duplicated on the display of the augmented reality device 820). In some implementations, the robotic system and/or the augmented reality device are used to facilitate insertion of the introducer access tools (e.g., introducer assembly 110 and/or introducer drill 440) but the robotic system 700, 800 and/or the augmented reality device 820 are not used for insertion of the other access tools or treatment devices (e.g., curved cannula assembly 210, steerable channeling instrument, treatment device 501). For example, the robotic system 700, 800 and/or augmented reality device 820 may only be used to facilitate access from a skin insertion location to a location within the cancellous bone portion of the vertebral body. In other implementations, the robotic system 700, 800 and/or augmented reality device 820 is used for all of the access and treatment steps.

In certain implementations, a sterile barrier can be formed between portions of the robotic system 800 and the patient. For example, in certain instances, a sterile drape is provided that is configured to maintain sterility and permit sterile placement of the surgical tools in a sterile field and further permits sterile removal of surgical tools for manual manipulations.

In accordance with several examples, any of the access or treatment instruments or devices described herein may include one or more sensors, trackers, or positioning markers (e.g., positioning beads, optical markers, GPS sensors) configured to facilitate detection or identification of the instruments by an existing commercial (e.g., non-proprietary) spinal robotic surgical guidance or navigation system instead of requiring a specifically-tailored and configured robotic system designed specifically for the particular access or treatment instruments. The positioning markers may be attached to or integral with the access or treatment instruments or devices. The spinal robotic guidance or navigation system may be adapted to detect and/or identify various instruments and to track or manipulate (e.g., control) the instruments.

Feedback from the robotic systems described herein may be combined with other input or information described herein to provide an optimum solution for a particular patient situation. For example, feedback determined from one or more imaging modalities indicative of pre-Modic change characteristics (e.g., vertebral endplate degeneration or endplate defects or multifidus muscle atrophy) or other conditions may be combined with feedback or information from the robotic systems to adjust treatment parameters or target regions. For example, PET scan, single photon emission CT, or MRI scan imaging may show an area of inflammation and MRI imaging may show regions indicative of existence or likelihood of pre-Modic change characteristics. This imaging input (along with other input described herein, such as information regarding biomarker activity or levels, nerve location based on nerve detection techniques, or other information) may be combined with feedback from the robotic systems to determine a desired target region within a vertebral body and/or desired ablation parameters (e.g., thermal dose, lesion size, shape, duration, temperature, etc.).

In accordance with several implementations, target, or candidate, vertebrae for treatment can be identified prior to treatment. The target, or candidate, vertebrae may be identified based on identification of various types of, or factors associated with, endplate degeneration and/or defects (e.g., focal defects, erosive defects, rim defects, corner defects, all of which may be considered pre-Modic change characteristics).

For example, one or more imaging modalities (e.g., MRI, CT, X-ray, fluoroscopic imaging) may be used to determine whether a vertebral body or vertebral endplate exhibits active Modic characteristics or “pre-Modic change” characteristics (e.g., characteristics likely to result in Modic changes, such as Type 1 Modic changes that include findings of inflammation and edema or type 2 Modic changes that include changes in bone marrow (e.g., fibrosis) and increased visceral fat content). For example, images obtained via MRI (e.g., IDEAL MRI) may be used to identify (e.g., via application of one or more filters) initial indications or precursors of edema or inflammation at a vertebral endplate prior to a formal characterization or diagnosis as a Type 1 Modic change.

Examples of pre-Modic change characteristics could include mechanical characteristics (e.g., loss of soft nuclear material in an adjacent intervertebral disc of the vertebral body, reduced disc height, reduced hydrostatic pressure, microfractures, focal endplate defects, erosive endplate defects, rim endplate defects, corner endplate defects, osteitis, spondylodiscitis, Schmorl's nodes) or bacterial characteristics (e.g., detection of bacteria that have entered an intervertebral disc adjacent to a vertebral body, a disc herniation or annulus tear which may have allowed bacteria to enter the intervertebral disc, inflammation or new capilarisation that may be caused by bacteria) or other pathogenetic mechanisms that provide initial indications or precursors of potential Modic changes or vertebral endplate degeneration or defects.

Accordingly, vertebral bodies may be identified as target candidates for treatment before Modic changes occur (or before painful symptoms manifest themselves to the patient) so that the patients can be proactively treated to prevent, or reduce the likelihood of, chronic low back pain before it occurs. In this manner, the patients will not have to suffer from debilitating lower back pain for a period of time prior to treatment. Modic changes may or may not be correlated with endplate defects and may or may not be used in candidate selection or screening. In accordance with several embodiments, Modic changes are not evaluated and only vertebral endplate degeneration and/or defects (e.g., pre-Modic change characteristics prior to onset or prior to the ability to identify Modic changes) are identified. Rostral and/or caudal endplates may be evaluated for pre-Modic changes (e.g., endplate defects that manifest before Modic changes that may affect subchondral and vertebral bone marrow adjacent to a vertebral body endplate).

The systems and methods described herein may also involve assessment of one or more biomarkers (e.g., biomarkers associated with pain, inflammation, or neurotransmission). Biomarkers may also be used to assess whether a particular subject is likely to be a candidate for nerve ablation treatment for treatment of back pain. For example, the biomarkers may be indicative of pre-Modic changes or symptoms likely to result in Modic changes or endplate damage (e.g., inflammation, edema, bone marrow lesions or fibrosis). The assessment of biomarker levels may indicate which vertebral bodies of a particular subject are candidates for treatment to prevent (or reduce the likelihood of) back pain from developing or worsening or to treat existing back pain. The pre-procedure biomarker assessment may also be combined with pre-procedure imaging. The biomarkers may include one or more of: an inflammatory cytokine (e.g., interleukins, interferons, tumor necrosis factors, prostaglandins, and chemokines), pain indicators (e.g., substance P, calcitonin gene-related peptides (CGRPs)), an edema factor, and/or other inflammatory factor. The biomarkers may be obtained, for example, from one or more blood serum samples (e.g., blood plasma). The biomarkers may be obtained over an extended period of time (e.g., a period of days, weeks, or months) or at a single instance in time. Biomarkers may also be identified in the images themselves and may be the tissue characteristics, bone marrow intensity changes, or other variations described above. Assessment of biomarkers may involve application of trained neural networks based on biomarker measurements of several (e.g., hundreds) of low back pain patients obtained previously and used to train the neural networks.

In some implementations, a level of biomarker(s) (e.g., substance P, cytokines, high-sensitivity C-reactive protein, or other compounds associated with inflammatory processes and/or pain and/or that correlate with pathophysiological processes associated with vertebral endplate degeneration or defects (e.g., pre-Modic changes) or Modic changes such as disc resorption, Type UI and Type IV collagen degradation and formation, or bone marrow fibrosis) may be obtained from a patient (e.g., through a blood draw (e.g., blood serum) or through a sample of cerebrospinal fluid) to determine whether the patient is a candidate for basivertebral nerve ablation treatment (e.g., whether they have one or more candidate vertebral bodies exhibiting factors or symptoms associated with endplate degeneration or defects (e.g., pre-Modic change characteristics)). Cytokine biomarker samples (e.g., pro-angiogenic serum cytokines such as vascular endothelial growth factor (VEGF)-C, VEGF-D, tyrosine-protein kinase receptor 2, VEGF receptor 1, intercellular adhesion molecule 1, vascular cell adhesion molecule 1) may be obtained from multiple different discs or vertebral bodies or foramina of the patient and compared with each other in order to determine the vertebral bodies to target for treatment. Other biomarkers may be assessed as well, such as neo-epitopes of type 111 and type IV pro-collagen (e.g., PRO-C3, PRO-C4) and type III and type IV collagen degradation neo-epitopes (e.g., C3M, C4M).

Biomarkers may include genetic markers, products of gene expression, autoantibodies, cytokine/growth factors, proteins or enzymes (such as heat shock proteins), and/or acute phase reactants. Biomarkers may include compounds correlated to back pain, such as inflammatory cytokines, Interleukin-1-beta (IL-1-beta), interleukin-1-alpha (IL-1-alpha), interleukin-6 (IL-6), IL-8, IL-10, IL-12, tumor necrosis factor-alpha (TNF-alpha), granulocyte-macrophage colony stimulating factor (GM-CSF), interferon gamma (INF-gamma), prostaglandin E2 (PGE2), aggrecan, proteoglycan, or glycosaminoglycans. Biomarkers may also be indicative of presence of tumor cells or tissue if tumor tissue is being targeted by the treatment. Biomarkers may be found in blood serum/plasma, urine, synovial fluid, tissue biopsy, foramina, intervertebral discs, cerebrospinal fluid, or cells from blood, fluid, lymph node, and/or tissue, including, for example, variations from a healthy intervertebral disc, such as aberrant pH levels or intervertebral disc damage, including, for example ruptured, degenerated, or prolapsed intervertebral discs, nucleus pulposus, fluid leaks or other damage that impinge on nerves that results in pain and/or inflammation. In some embodiments, the biomarkers can be indicators identified from images.

In some implementations, samples are obtained over a period of time and compared to determine changes in levels over time. For example, biomarkers may be measured weekly, bi-monthly, monthly, every 3 months, or every 6 months for a period of time and compared to analyze trends or changes over time. If significant changes are noted between the biomarker levels (e.g., changes indicative of endplate degeneration or defects (e.g., pre-Modic change characteristics) or Modic changes, as described above), treatment may be recommended and performed to prevent or treat back pain.

Biomarker levels (e.g., substance P, cytokine protein levels, PRO-C3, PRO-C4, C3M, C4M levels, aberrant pH levels or intervertebral disc damage) may be measured using various in vivo or in vitro kits, systems, and techniques (e.g., radio-immunoassay kits/methods, enzyme-linked immunosorbent assay kits, immunohistochemistry techniques, array-based systems, bioassay kits, in vivo injection of an anticytokine immunoglobulin, multiplexed fluorescent microsphere immune-assays, homogeneous time-resolved fluorescence assays, bead-based techniques, interferometers, flow cytometry, etc.). Cytokine proteins may be measured directly or indirectly, such as by measuring mRNA transcripts.

In accordance with several implementations, biomarkers may be used to confirm treatment efficacy (e.g., whether the procedure resulted in effective ablation of a basivertebral nerve within a vertebral body or an intraosseous nerve within another bone and achieved a desirable therapeutic response). Biomarkers can include anatomical, physiological, biochemical, molecular parameters or imaging features that can be used to confirm treatment efficacy. Biomarkers can be detected and measured by a variety of methods, including but not limited to, physical examination, laboratory assays (such as blood samples), and medical imaging. Biomarkers may be obtained via biological tissue sampling or in a minimally invasive manner (e.g., from blood, saliva, cerebrospinal fluid, or urine). Tissue imaging may also be used to detect and measure biomarkers.

The measurement of biomarker levels can utilize one or more capture or detection agents that specifically bind to the biomarker, such as a labeled antibody to bind and detect a biomarker. In some implementations, measurement of biomarkers may utilize a detection agent that has a functional interaction with the biomarker. In other implementations, measurement of biomarkers may be carried out using imaging/spectroscopy techniques that allow biomarker levels to be assessed in a non-invasive manner or by tissue sampling. Capture or detection agents may be used. In some implementations, binding of a biomarker to a capture agent and/or interaction of the biomarker with a detection agent results in a quantitative, or detectable, signal. The signal may include, for example, a colorimetric, fluorescent, heat, energy, or electric signal. The detectable, quantitative signal may be transmitted to an external output or monitoring device. In some implementations, binding of a biomarker to a capture agent results in a signal that can be transmitted to an external monitoring device. For example, binding of a biomarker to a capture or detection agent may be detected using a high sensitivity fluorescence technique such as a resonance energy transfer method (e.g., Forster resonance energy transfer, bioluminescence resonance energy transfer, or surface plasmon resonance energy transfer).

In various implementations, the measurement of pre- and post-treatment biomarker levels may be carried out using the same device that is used to carry out the treatment (e.g., ablation, denervation) or a component attached to the treatment device. Alternatively, biomarker level or activity may be carried out using a separate device from the treatment device. The separate biomarker assessment device may be inserted through the same introducer as the treatment device or a separate introducer.

One or more samples, images, and/or measurements may be obtained from a patient prior to treatment and after treatment and the presence of one or more biomarkers in the pre-treatment and post-treatment samples may be compared to confirm treatment efficacy. The comparison may involve comparison of levels or activity of the biomarkers within the samples. For example, there may be a burst or spike in biomarker concentration following ablation of the basivertebral nerve trunk or branches thereof that can be detected or measured within a collected biological sample.

As another example, the change in the level or activity of the biomarker(s) may be an indirect response to ablation of the basivertebral nerve trunk or branches thereof (e.g., an inflammatory or anti-inflammatory protein, such as a cytokine protein, a heat shock protein, or a stress response protein that is triggered in response to ablative energy being applied to the target treatment region or a non-protein biomarker associated with nervous activity, such as catecholamines, neurotransmitters, norepinephrine levels, neuropeptide Y levels, epinephrine levels, and/or dopamine levels). The post-treatment samples may be obtained immediately following treatment (e.g., within seconds after treatment, within about 15 minutes following treatment, or within about 30 minutes following treatment) and/or may be obtained after a more significant amount of time following treatment (e.g., 24 hours after treatment, 3 days after treatment, 1 week after treatment, 2 weeks after treatment, 1 month after treatment, 3 months after treatment, 6 months after treatment).

Various inputs (e.g., biomarker activity or levels, physiological parameter measurements indicative of neuronal activity, temperature measurements, impedance measurements, and/or images), may be combined (e.g., weighted combinations) to generate a quantitative pain score that can be used to confirm pain relief (as an adjunct or as an alternative to subjective pain relief confirmation). The pain score may be generated using an automated algorithm executed by a processor of a pain analyzer system. The pain analyzer system may receive input from various sensors, imaging devices, and/or the like and the input may be weighted and/or processed by one or more circuits or processing modules of the pain analyzer system to generate the quantitative pain score. The quantitative pain score may be output on a display (e.g., of a generator).

### CONCLUSION

In some implementations, the system comprises various features that are present as single features (as opposed to multiple features). For example, in one embodiment, the system includes a single radiofrequency generator, a single introducer cannula with a single stylet, a single radiofrequency energy delivery device or probe, and a single bipolar pair of electrodes. A single thermocouple (or other means for measuring temperature) may also be included. Multiple features or components are provided in alternate embodiments.

In some implementations, the system comprises one or more of the following: means for tissue modulation (e.g., an ablation or other type of modulation catheter or delivery device), means for monitoring temperature (e.g., thermocouple, thermistor, infrared sensor), means for imaging (e.g., MRI, CT, fluoroscopy), means for accessing (e.g., introducer assembly, curved cannulas, drills, curettes), means for actuating (e.g., threaded knob or screw actuation mechanism, sliding actuator, pullwire actuator, lever, hydraulic actuator, pneumatic actuator, electrical actuator, push button actuator, mechanical linear actuator, etc.)

Although certain embodiments and examples have been described herein, aspects of the methods and devices shown and described in the present disclosure may be differently combined and/or modified to form still further embodiments. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. The section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying thermal energy” include “instructing the applying of thermal energy.”

The terms “top,” “bottom,” “first,” “second,” “upper,” “lower,” “height,” “width,” “length,” “end,” “side,” “horizontal,” “vertical,” and similar terms may be used herein; it should be understood that these terms have reference only to the structures shown in the figures and are utilized only to facilitate describing embodiments of the disclosure. The terms “proximal” and “distal” are opposite directional terms. For example, the distal end of a device or component is the end of the component that is furthest from the operator during ordinary use. A distal end or tip does not necessarily mean an extreme distal terminus. The proximal end refers to the opposite end, or the end nearest the operator during ordinary use. Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers preceded by a term such as “about” or “approximately” include the recited numbers. For example, “about 2:1” includes “2:1.” For example, the terms “approximately”, “about”, and “substantially” as used herein represent an amount close to the stated amount that still performs a desired function or achieves a desired result.

What is claimed is:

1. A method of ablating a basivertebral nerve within a vertebral body, the method comprising:
providing a desired trajectory to access a target treatment site within the vertebral body to a computer-based control system of a robotic system comprising one or more robotic arms;
coupling a bone access tool to the one or more robotic arms of the robotic system;
controlling insertion of the bone access tool through skin adjacent the vertebral body and into the vertebral body using the robotic system, the bone access tool comprising an introducer cannula,
wherein controlling insertion of the bone access tool comprises use of an augmented reality device that includes a see-through optical head mounted display and use of one or more input devices communicatively coupled to the robotic system;
inserting a radiofrequency energy delivery device through the introducer cannula to a target treatment site within the vertebral body; and
applying radiofrequency energy to the target treatment site using the radiofrequency energy delivery device sufficient to ablate the basivertebral nerve.

2. The method of claim 1, wherein the bone access tool comprises a marker to facilitate registration of the bone access tool with a tracking system of the robotic system.

3. The method of claim 1, wherein the augmented reality device comprises a headset or eyewear.

4. The method of claim 1, wherein the one or more input devices comprise one or more joysticks.

5. The method of claim 4, wherein the one or more joysticks are communicatively coupled to the augmented reality device.

6. The method of any one of claim 1, wherein the desired trajectory is displayed as a virtual image on the display of the augmented reality device.

7. The method of claim 1, wherein the desired trajectory is configured to end at a region that includes a basivertebral nerve trunk.

8. The method of any one of claims 1 to 7, wherein the desired trajectory is determined based on pre-operative images of spinal anatomy surrounding and including the vertebral body.

9. The method of any one of claims 1 to 7, wherein the desired trajectory is determined based on particular characteristics of the vertebral body.

10. The method of claim 9, wherein the characteristics include bone structure characteristics.

11. The method of claim 10, wherein the bone structure characteristics include bone density.

12. The method of claim 9, wherein the characteristics include an anatomical identification of the vertebral body by vertebral level and number.

13. The method of any one of claims 1 to 7, wherein the one or more robotic arms have at least six degrees of freedom.

14. The method of any one of claims 1 to 7, wherein the robotic system comprises one or more imaging devices configured to provide feedback to the robotic system to control said insertion of the bone access tool.

15. The method of any one of claims 1 to 7, wherein controlling said insertion of the bone access tool comprises responding to haptic feedback provided by the robotic system.

16. The method of claim 1, wherein:
the bone access tool comprises a marker to facilitate registration of the bone access tool with a tracking system of the robotic system;
the augmented reality device comprises a headset or eyewear;
the desired trajectory is displayed as a virtual image on the display of the augmented reality device;
the desired trajectory is determined based on pre-operative images of spinal anatomy surrounding and including the vertebral body; and
the desired trajectory is determined based on particular characteristics of the vertebral body.

17. A robotic system for facilitating intraosseous nerve ablation comprising:
an operator control console comprising a computer-based control system including at least one processor that is configured to execute program instructions stored on a non-transitory computer-readable medium to carry out an intraosseous nerve ablation procedure to ablate an intraosseous nerve within a vertebral body;
one or more robotic surgical arms configured to move with six or more degrees of freedom and to support or carry access tools or treatment devices;
a tracking system that can be used to capture a position of at least a portion of a patient, the access tools or treatment devices, and the one or more robotic surgical arms; and
a display, wherein the computer-based control system is configured to display a desired trajectory directed to a target treatment site within the vertebral body to perform the intraosseous nerve ablation procedure.

18. The system of claim 17, wherein the computer-based control system uses a closed-loop system to control automated insertion of the access tools or treatment devices.

19. The system of claim 18, wherein the closed-loop system incorporates feedback based on one or more trained neural networks.

20. The system of claim 17, wherein the computer-based control system is configured to receive an input from a user via the operator control console to modify the desired trajectory directed to the target treatment site.

21. The system of claim 17, wherein the display comprises a see-through display on an augmented reality headset or eyewear and wherein the system is configured for a user to operate in an augmented reality environment.

22. The system of any one of claims 17 to 21, wherein the one or more robotic surgical arms are carried by a mobile cart.

23. The system of any one of claims 17 to 21, further comprising a surgical instrument guide configured to be coupled to the one or more robotic surgical arms to facilitate guided insertion of the access tools or treatment devices along the desired trajectory.

24. The system of any one of claims 17 to 21, wherein the operator control console comprises an augmented reality device.

25. A method of ablating an intraosseous nerve within a vertebral body of a patient, the method comprising:
positioning, with a robotically-controlled system, a surgical instrument guide along a trajectory directed to a target treatment site within the vertebral body;
inserting an access assembly through the surgical instrument guide and into the vertebral body;
inserting a radiofrequency energy delivery device through the access assembly to the target treatment site within the vertebral body; and
applying radiofrequency energy to the target treatment site using the radiofrequency energy delivery device sufficient to ablate the intraosseous nerve.

26. The method of claim 25, wherein the trajectory is determined automatically by the robotically-controlled system based on: (i) pre-operative images of a vertebra corresponding to the vertebral body and surrounding patient anatomy; (ii) a location of the vertebral body; and (iii) patient-specific characteristics associated with the vertebral body.

27. The method of claim 26, wherein the pre-operative images comprise magnetic resonance images or computed tomography images of a portion of the patient's spine.

28. The method of claim 26, wherein the location of the vertebral body includes an identification of whether the vertebral body is of a lumbar vertebra or a sacral vertebra and a particular level of the vertebra.

29. The method of claim 26, wherein the patient-specific characteristics comprise a bone density measurement.

30. The method of claim 25, wherein the robotically-controlled system comprises one or more robotic arms.

31. The method of claim 30, wherein the one or more robotic arms have at least six degrees of freedom.

32. The method of claim 25, wherein the robotically-controlled system comprises an operator control console comprising at least one processor.

33. The method of claim 30, wherein the robotically-controlled system comprises one or more imaging devices configured to provide feedback to the robotically-controlled system to control insertion of the access assembly and/or the radiofrequency energy delivery device.

34. The method of claim 33, wherein the one or more imaging devices are carried by the one or more robotic arms.

35. The method of any one of claims 25 to 34, wherein the robotically-controlled system uses a closed-loop system to control automated adjustments to the insertion of the access assembly and/or the radiofrequency energy delivery device.

36. The method of claim 35, wherein the closed-loop system incorporates feedback based on one or more trained neural networks.

37. The method of any one of claims 25 to 34, further comprising receiving instructions from a user modifying a desired trajectory directed to the target treatment site.

38. The method of any one of claims 25 to 34, further comprising manually inserting the access assembly through the surgical instrument guide and into the vertebral body and/or manually inserting a radiofrequency energy delivery device through the access assembly to the target treatment site within the vertebral body.

39. The method of any one of claims 25 to 34, further comprising automatically adjusting, via the robotically-controlled system, a position of the surgical instrument guide based on a change in position of the vertebral body such that a spatial relationship between the surgical instrument guide and the vertebral body remains substantially unaltered as at least a portion of an operation of ablating the intraosseous nerve is performed.

40. The method of any one of claims 25 to 34, wherein the trajectory is configured to end at a region that includes a basivertebral nerve trunk.

41. The method of claim 25, wherein the trajectory is determined by a user based on pre-operative images of at least the vertebral body.

42. A system for facilitating nerve ablation comprising:
an operator control console comprising a computer-based control system including at least one processor that is configured to execute program instructions stored on a non-transitory computer-readable medium to carry out a nerve ablation procedure to ablate an intraosseous nerve within one or more vertebral bodies;
a robotic surgical arm configured move with at least three degrees of freedom and to support a surgical instrument guide;
a tracking system to capture a position of the robotic surgical arm; and
an access assembly and/or a radiofrequency energy delivery device configured to be inserted through the surgical instrument guide.

43. The system of claim 42, wherein the computer-based control system is configured to provide feedback to a user to control insertion of the access assembly and/or the radiofrequency energy delivery device.

44. The system of claim 42, wherein the computer-based control system uses a closed-loop system to control insertion of the access assembly and/or the radiofrequency energy delivery device.

45. The system of any one of claims 42 to 44, wherein the computer-based control system is configured to display on a display a desired trajectory directed to a target treatment site within a vertebral body to a user of the system.

46. The system of claim 45, wherein the desired trajectory is displayed as a virtual image on the display of an augmented reality device.

47. The system of claim 45, wherein the desired trajectory is configured to end at a region that includes a basivertebral nerve trunk.

48. The system of claim 45, wherein the desired trajectory is determined based on pre-operative images of spinal anatomy surrounding and including the vertebral body.

49. The system of claim 45, wherein the desired trajectory is determined based on particular characteristics of the vertebral body.

50. The system of claim 49, wherein the characteristics include bone structure characteristics.

51. The system of claim 50, wherein the bone structure characteristics include bone density.

52. The system of claim 49, wherein the characteristics include an anatomical identification of the vertebral body by vertebral level and number.

53. The system of any one of claims 42 to 44, wherein the robotic surgical arm has at least six degrees of freedom.

54. A method of facilitating nerve ablation in a patient comprising:
generating, by a computer system comprising at least one processor, a virtual trajectory,
wherein the virtual trajectory is a virtual axis,
wherein the virtual axis is a three-dimensional digital representation indicating a position, orientation, or combination thereof, for advancing one or more physical surgical tools or physical surgical instruments towards a treatment site; and
displaying the position, orientation, or combination thereof, using the display, of the virtual surgical axis onto a representation of a portion of the patient anatomy so as to superimpose the virtual trajectory onto patient's anatomy,
wherein the display of the position, orientation, or combination thereof, of the virtual axis is configured to be maintained by the computer system in relationship to one or more anatomic structures when the patient moves,
wherein the one or more anatomic structures are registered in a coordinate system, and
wherein the virtual axis is registered in the coordinate system.

55. The method of claim 54, further comprising displaying the virtual axis with a see-through optical head mounted display that is registered in the coordinate system.

56. The method of claim 54, further comprising inserting a surgical tool along the virtual trajectory and into a vertebral body of the patient and ablating a basivertebral nerve trunk within the vertebral body of the patient.

57. A system for facilitating nerve ablation in a patient, the system comprising:
at least one processor,
at least one display, and
at least one user interface,
wherein the system is configured to generate a virtual trajectory,
wherein the virtual trajectory is a virtual axis,
wherein the virtual axis is a three-dimensional digital representation indicating desired insertion path of a plurality of instruments towards a target site within a vertebral body, and
wherein the target site corresponds to a location of a basivertebral nerve trunk within the vertebral body.

58. A method of modulating a basivertebral nerve within a vertebral body, the method comprising:
inserting an access assembly within a vertebral body using a robotically-controlled system, the access assembly including a cannula;
inserting an ablation device through the cannula to a target treatment site within the vertebral body using a robotically-controlled system; and
applying treatment to the target treatment site using the ablation device sufficient to ablate the basivertebral nerve.

59. The method of claim 58, wherein the ablation device comprises a radiofrequency energy delivery device, an ultrasound energy delivery device, a microwave energy delivery device, a laser energy delivery device, a thermal energy delivery device, a fluid-based ablation device, a cryoablation device, or a chemical ablation device.
